(19) United States
(12) Patent Application Publication
(10) Pub. No.: US 2023/0111925 A1
(43) Pub. Date: Apr. 13, 2023

(54) FIBROTIC DISEASE MECHANISM AND THERAPEUTIC DRUG THEREFOR

(71) Applicant: GUANGZHOU WOMEN AND CHILDREN'S MEDICAL CENTER, Guangzhou, Guangdong (CN)

(72) Inventors: Yuxia ZHANG, Guangzhou, Guangdong (CN); Yanhui XU, Guangzhou, Guangdong (CN); Bing HUANG, Guangzhou, Guangdong (CN); Ming LIU, Guangzhou, Guangdong (CN); Zhiyao ZHAO, Guangzhou, Guangdong (CN); Jun WANG, Guangzhou, Guangdong (CN); Zhanghua CHEN, Guangzhou, Guangdong (CN); Rongli FANG, Guangzhou, Guangdong (CN)

(73) Assignee: GUANGZHOU WOMEN AND CHILDREN'S MEDICAL CENTER, Guangzhou, Guangdong (CN)

(21) Appl. No.: 17/798,477

(22) PCT Filed: Feb. 9, 2021
(86) PCT No.: PCT/CN2021/076307

§ 371 Date: Aug. 9, 2022

(30) Foreign Application Priority Data
Feb. 10, 2020 (CN) 202010084911.2

(51) Int. Cl. A61K 31/519; A61K 31/444; A61K 31/4709; A61K 31/496; A61K 31/44; A61K 31/7048; A61P 1/00; A61P 1/16; A61P 19/04; A61P 11/00

(52) U.S. Cl. CPC A61K 31/519; A61K 31/444; A61K 31/4709; A61K 31/496; A61K 31/44; A61K 31/7048; A61P 1/00; A61P 1/16; A61P 19/04; A61P 11/00

(57) ABSTRACT

The present invention relates to a fibrotic disease mechanism, and a preventive/therapeutic drug and method therefor. The present invention can improve the level of cAMP by means of a PDE inhibitor such as dipyridamole to treat fibrotic diseases and inhibit the progress of fibrosis. The present invention further achieves anti-inflammatory and immune regulation effects, and achieves a therapeutic effect on all aspects of the occurrence and development of fibrosis.

### TECHNICAL FIELD

The present disclosure relates to a fibrotic disease mechanism, a therapeutic method and drug use thereof.

### BACKGROUND

Fibrosis is characterized by the increase of fibrillar connective tissues and the decrease of parenchymal cells in organs and tissues, which can occur in a variety of organs. Continuous progress of fibrosis can lead to destruction of organ structure, functional decline and even failure, which seriously threatens human health and life. Worldwidely, tissue fibrosis is a major cause of disability and death in many diseases and plays an important role in the occurrence and development of diseases in major organs of a human body, such as liver, cardiovascular, lung, kidney, etc.

Specifically, fibrosis may appear in hepatobiliary diseases, such as cirrhosis, hepatic fibrosis, liver injury and biliary atresia (sometimes referred to as BA hereafter), etc. BA is a common disease causing obstructive jaundice in infants. At a perinatal period (for example, from 28 weeks of gestation to 4 weeks after birth), the infants may be stimulated by virus infection and other factors to cause autoimmune reaction of pathological changes such as epithelium apoptosis or necrosis of the biliary duct, bile duct injury, inflammation and fibrosis, with poor prognosis and high mortality. The basic pathological changes of biliary atresia, such as progressive inflammation of intrahepatic and extrahepatic bile ducts and hepatic fibrosis, and this type of hepatic fibrosis developed faster and more aggressive than other adult diseases. Although Kasai operation can partially relieve symptoms of extrahepatic biliary obstruction and delay the progress of the disease, most child patients still have progressive development due to postoperative intrahepatic bile duct inflammation, which eventually leads to cirrhosis, portal hypertension and even hepatic failure. Therefore, the biliary atresia becomes a serious disease that endangers lives of the child patients.

The incidence rates of gastrointestinal diseases, such as inflammatory bowel disease, undifferentiated (also referred to as indeterminate) colitis, Crohn's disease (hereinafter sometimes referred to as CD) and Ulcerative colitis (hereinafter sometimes referred to as UC) are increasing year by year all over the world, and these diseases are also common clinical diseases in China. Fibrosis may also be found in gastrointestinal diseases. Gastrointestinal diseases lead to acute attacks, persistent chronic subclinical inflammatory reactions or repeated attacks of diseases, which seriously affect the health and growth of the majority of patients, especially child patients, and bring huge economic burden to families and society.

Lung diseases, for example, pulmonary fibrosis, silicosis, pulmonary hypertension and related diseases. Pulmonary fibrosis seriously affects human respiratory function, which is characterized by tussiculation and progressive dyspnea. With the aggravation of the disease and lung injury, the respiratory function of patients continues to deteriorate. Idiopathic pulmonary fibrosis (IPF) is a fatal disease with short survival time and few treatment options, and is a basically irreversible disease. The fibrosis of IPF is characterized by the existence of activated fibroblasts, which produces excessive fibrous substances to destroy alveolar structure. Incidence and mortality of idiopathic pulmonary fibrosis are increasing year by year.

For a long time, the pathogenesis mechanisms of fibrotic diseases are not clear and these diseases are difficult to treat.

### SUMMARY

In order to solve the above problems, the inventors made an in-depth study on occurrence mechanisms of various fibrotic diseases, dominant cells causing this immunoreaction and molecular mechanisms thereof, and obtained the following results. The present disclosure can increase the level of cyclic adenosine monophosphate (hereinafter sometimes referred to as cAMP) by administering a phosphodiesterase inhibitor (hereinafter referred to as PDE inhibitor) to treat the fibrotic diseases and inhibit the progress of fibrosis. Meanwhile, it was found by the inventors that PDE inhibitor also has anti-inflammatory and immunomodulatory effects, and has therapeutic effects on all aspects of the occurrence and development of fibrosis.

It was found by the inventors that in patients with hepatic fibrosis, such as biliary atresia patients, all immune cell subtypes in liver tissue express phosphodiesterase (hereinafter sometimes referred to as PDE), especially PDE4B. In this way, it was found by the inventors that inhibiting the activity of PDE could improve the inhibited cAMP signalling pathways in liver injury, increase the level of cAMP in liver, improve immune environment, resist inflammation and inhibit fibrosis, thus playing a protective role in liver. It was found by the inventors that using phosphodiesterase inhibitors (such as Dipyridamole (hereinafter sometimes referred to as Dip)) could significantly inhibit the expression of fibroblast-related genes in cell experiments in vitro, and could protect virus-infected mice from developing biliary atresia in animal models of RRV-induced biliary atresia. Therefore, using PDE inhibitors to relieve the inhibition of cAMP pathways caused by biliary atresia, plays an important role in delaying liver injury and fibrosis. PDE inhibitors (such as Dipyridamole) may be used to treat and/or prevent the occurrence and development of hepatobiliary diseases such as biliary atresia.

It was also found by the inventors that in patients with gastrointestinal tract diseases, such as various enteritis, there are defects such as infiltration of highly inflammatory macrophages, CD39⁺IET deficiency and platelet aggregation, and the defective cAMP reaction pathway acts as a common mechanism. It was found by the inventors that inhibiting the activity of PDE, for example, administering PDE inhibitors (such as Dipyridamole) could improve the inhibited cAMP signalling pathways in gastrointestinal tract diseases, improve the immune environment of gastrointestinal tract, resist inflammation, and meanwhile, also inhibit gastrointestinal tract fibrosis, thus providing a new treatment solution for the prevention and treatment of gastrointestinal tract diseases.

It was further found by the inventors that in lung disease, PDE inhibitors (such as Dipyridamole) could promote type I interferon signals and alleviate lung injury caused by viral infection. In cell test in vitro, the addition of PDE inhibitors such as Dipyridamole significantly promoted mRNA and protein levels of IFN-β in cells; in the mouse model, Dipyridamole treatment could significantly reduce the infiltration of inflammatory cells in the lungs of mice and alleviate the alveolar injury. These results all indicate that PDE inhibitors (such as Dipyridamole) are effective in preventing and treating pulmonary inflammation, immunomodulation and fibrosis.

As described above, the present disclosure relates to the following aspects.

[1]. A method of preventing and/or treating a fibrotic disease, comprising: administering a PDE inhibitor to a subject.

[2]. The method of [1] above, the PDE inhibitor is at least one selected from the group consisting of PDE1, PDE2, PDE3, PDE4, PDE5, PDE6, PDE7, PDE8, PDE9, PDE10, and PDE11 inhibitors.

[3]. The method of any one of [1] to [2] above, the PDE inhibitor is a pan PDE inhibitor.

[4]. The method of any one of [1] to [3] above, the PDE inhibitor is Dipyridamole.

[5]. The method of any one of [1] to [4] above, the fibrotic disease is selected from fibrotic diseases of liver, gallbladder, lung, kidney, bladder, heart, blood vessel, eye, skin, pancreas, gastrointestinal, bone marrow, penis, breast, and muscle.

[6]. The method of any one of [1] to [5] above, the fibrotic disease is selected from fibrotic diseases of liver, gallbladder, lung and gastrointestinal.

[7]. The method of any one of [1] to [6] above, the fibrotic disease is selected from cirrhosis, hepatic fibrosis, liver injury, hepatic failure and biliary atresia.

[8]. The method of any one of [1] to [7] above, the fibrotic disease is selected from idiopathic pulmonary fibrosis, silicosis, cystic fibrosis and pulmonary hypertension.

[9]. The method of any one of [1] to [8] above, the fibrotic disease is selected from fibrosis of stomach, duodenum, small intestine or colon, for example, colitis, undifferentiated colitis, Crohn's disease, ulcerative colitis, chronic colitis and chronic eosinophilic colitis.

[10]. Use of a PDE inhibitor in preparation of a medicament for preventing and/or treating a fibrotic disease.

[11]. The use of [10] above, the PDE inhibitor is at least one selected from the group consisting of PDE1, PDE2, PDE3, PDE4, PDE5, PDE6, PDE7, PDE8, PDE9, PDE10, and PDE11 inhibitors.

[12]. The use of any one of [10] to [11] above, the PDE inhibitor is a pan PDE inhibitor.

[13]. The use of any one of [10] to [12] above, the PDE inhibitor is Dipyridamole.

[14]. The use of any one of [10] to [13] above, the fibrotic disease is selected from fibrotic diseases of liver, gallbladder, lung, kidney, bladder, heart, blood vessel, eye, skin, pancreas, gastrointestinal, bone marrow, penis, breast, and muscle.

[15]. The use of any one of [10] to [14] above, the fibrotic disease is selected from fibrotic diseases of liver, gallbladder, lung and gastrointestinal.

[16]. The use of any one of [10] to [15] above, the fibrotic disease is selected from cirrhosis, hepatic fibrosis, liver injury, hepatic failure and biliary atresia.

[17]. The use of any one of [10] to [16] above, the fibrotic disease is selected from idiopathic pulmonary fibrosis, silicosis, cystic fibrosis and pulmonary hypertension.

[18]. The use of any one of [10] to [17] above, the fibrotic disease is selected from fibrosis of stomach, duodenum, small intestine or colon, for example, colitis, undifferentiated colitis, Crohn's disease, ulcerative colitis, chronic colitis and chronic eosinophilic colitis.

[19]. A combination drug for the fibrotic disease of any one of [1] to [9] above, comprising a combination of the PDE inhibitor of any one of [10] to [18] above and other active ingredients, and a pharmaceutically acceptable carrier.

[20]. A pharmaceutical composition, comprising the PDE inhibitor of any one of [10] to [18] above, and a pharmaceutically acceptable carrier.

[21]. The use of any one of [1] to [18] above, the PDE inhibitor is at least one selected from the group consisting of ITI214, PF-05085727, PF-04447943, Milirinone, Cilostazol, Vesnarinone, Roflumilast, Icariin, and Mardepodect hydrochloride.

[22]. The combination drug of [19] above, the PDE inhibitor is at least one selected from the group consisting of ITI214, PF-05085727, PF-04447943, Milirinone, Cilostazol, Vesnarinone, Roflumilast, Icariin, and Mardepodect hydrochloride.

[23]. The pharmaceutical composition of [20] above, the PDE inhibitor is at least one selected from the group consisting of ITI214, PF-05085727, PF-04447943, Milirinone, Cilostazol, Vesnarinone, Roflumilast, Icariin, and Mardepodect hydrochloride.

### BRIEF DESCRIPTION OF THE DRAWINGS

FIG. 1 shows that all immune cell subtypes in liver tissues of patients with biliary atresia express PDE, especially PDE4B. According to results of single-cell sequencing in the livers of the patients with biliary atresia, it is found that various immune cell subtypes in the livers of the patients with biliary atresia express various types of PDE (referring to FIG. 1A), especially PDE4B is more widely expressed in various immune cells, suggesting that inhibiting the activity of PDE can relieve the inhibition of cAMP pathway and improve the level of cAMP in the liver, thus playing a protective role in the liver.

FIG. 2 shows that a pan PDE inhibitor Dipyridamole (Dip) inhibits the expression of fibroblast genes. After the hepatic stellate cell line LX-2 cultured in vitro being stimulated by Dip, qPCR results show that the basic levels of fibrosis-related genes α-SMA, COLL1A1, COLL1A2 and COLL3A1 are significantly inhibited; and after being activated by cytokine TGF-β, the expression of the above genes is also inhibited by Dip (see FIG. 2A). In FIG. 2A, the bargraphs in each group of histograms are Nip (control), Dip, TGF-β and TGF-β+Dip from left to right.

FIG. 3 shows that Dipyridamole (Dip) protects virus-infected mice from developing biliary atresia in the models of biliary atresia caused by RRV infection in newborn mice. After administration with Dip, the weight of RRV-infected mice increases significantly (A) (in FIG. A, the weight gain curve of RRV group is at the bottom, the weight gain curve of RRV+Dip group is at the middle, and the weight gain curve of the control group is at the top), and there is no jaundice (B), suggesting that Dip can prevent RRV-induced biliary atresia. HE staining of liver tissues show that liver necrosis and infiltration lesions of inflammatory cells decrease in the Dip group (C), while sinus red staining indicates that liver fiber staining decreases significantly (D). Therefore, Dip protects liver injury and liver fibrosis caused by RRV virus infection.

FIG. 4 shows that Dipyridamole (Dip) inhibits the replication of RRV virus in livers in the biliary atresia models caused by RRV infection in newborn mice. Non-structural protein 3 (NSP3) is involved in virus replication in vivo. The qPCR results of mouse liver tissues suggest that the level of NSP3 in the mice of the Dip group is significantly lower than that in the non-drug group (A), which indicates that Dip can inhibit the invasion and toxicity of virus to liver and protect liver.

FIG. 5 shows that after administration with Dip, the infiltration of inflammation-related cells such as neutrophils and monocytes in the livers of RRV mice decreases (A), and the mRNA levels of inflammatory factors TNF-α and IL-1β decrease significantly (B). All these results suggest that Dip can inhibit the liver inflammation caused by RRV virus infection.

FIG. 6 shows that in the models of liver injury caused by bile duct ligation (BDL), administration of DIP to the mice can significantly improve the liver injury caused by BDL, which shows that DIP can slow down a continuous weight loss caused by BDL, and can also significantly reduce a liver weight ratio (A). In addition, an increased liver function index ALT is also significantly reduced in the mice administered with DIP (B). It is found by further analysis that DIP can reduce the level of inflammatory cytokines IFN-γ in liver (C). ELISA results show that the levels of inflammatory cytokines such as TNFα and IFN-β in plasma are significantly reduced in BDL mice treated with DIP (D). The livers of BDL model are accompanied by increase of fibroblasts, destruction of liver tissue structures and infiltration of inflammatory cells, which are all improved by treatment with DIP. Immunohistochemical results suggest that in BDL mice treated with DIP, fibroblasts are significantly reduced, liver tissue structures are clear, and inflammatory cells are reduced (E). These results all indicate that the PDE inhibitor DIP can inhibit liver inflammation and injury caused by cholestasis and protect the liver.

FIG. 7 shows Dipyridamole (Dip) promoting type I interferon signals and alleviating lung injury caused by virus infection. (A) DIP and DMSO are respectively added to lung epithelial cells A549, and the cells are infected with SEV (MOI=1) at the same time, then cells and culture supernatants at 0, 16 and 24 hours are collected respectively. Total RNA is extracted from cells, and the mRNA level of IFN-β is detected by fluorescence quantitative PCR. The level of IFN-β in the supernatants is detected by ELISA. Addition of Dip significantly promotes the mRNA and protein levels of IFN-β in cells. In FIG. A, in each group of histograms, the left bargraph represents the DMSO group and the right bargraph represents the DIP group. (B) Different doses of DIP (0 mM, 4 mM, 20 mM) are respectively added to 293T cells, and the cells are infected with SeV (MOI=1), then the cells are collected, and phosphorylation levels of kinase TBK1 and transcription factor IRF3 are detected by western blot. (C) Different PDE inhibitors (hereinafter sometimes referred to as PDEi) (DIP and the like) (5 μM) and DMSO are added to lung epithelial cells A549 respectively, and the cells are infected with SEV (MOI=1) for 24 hours at the same time. Total RNA is collected from the cells, and the mRNA level of IFN-β is detected by fluorescence quantitative PCR. PDEi can significantly up-regulate the mRNA level of the type I interferon IFN-β. (D) A mouse model infected by RNA virus VSV is constructed according to the methods known in the art. The mice are injected with Dipyridamole (30 mg/kg) intraperitoneally from day −3 and injected with VSV (10⁸ PFU/g) through tail vein on day 0 and day 4, and the administrating lasts for 7 days. H&E staining of mouse lung shows that the infiltration of pulmonary inflammation in lung and alveolar injury are significantly reduced after DIP treatment.

FIG. 8 shows Dipyridamole alleviates proliferation of fibroblasts of colitis in mice and clinical experiments. (A) Mice are divided into three groups, and the control group is fed with normal diet and drinking water; the cDSS+DIP (chronic colitis treated with DIP) group is injected intraperitoneally with 100 uL of DIP (50 mk/kg) from day −2, twice a day, which lasts until day 28; and the cDSS+vehicle (chronic colitis control) group is injected intraperitoneally with the same volume of control vehicle from day −2, twice a day, which also lasts until day 28. Meanwhile, in the cDSS+DIP and cDSS+vehicle groups, the normal drinking water is replaced to drinking water containing 2% dextran sulfate sodium (DSS) on days 0-7 and days 21-28, and the normal drinking water is provided at the rest of the time. The above-mentioned cDSS is chronic dextran sulfate sodium, which refers to the establishment of chronic colitis model by adding 2% dextran sodium sulfate (DSS) into the drinking water of mice. In all groups, on day 28, mice colons are taken for immunofluorescence staining of frozen sections. (B) shows colon immunofluorescence from (A), wherein the blue refers to nucleus, and the red refers to fibroblast indexes (above: COL1A2; below: CD90). The results indicates that after DIP injection, the number of fibroblasts in colon of mice with chronic colitis decreases significantly. Control: N=3; cDSS+DIP (chronic colitis treated with DIP) group: N=3; and cDSS+vehicle (chronic colitis control) group: N=3. (C) In the preliminary clinical experiment of DIP, the inventors divide child patients into three groups: the control group (normal colon) (N=4), DIP-Before and DIP-after (before and after DIP treatment) (N=7, Colitis is chronic colitis, N=3, EOS is chronic eosinophilic colitis, N=3, and IBDu is undifferentiated inflammatory bowel disease, N=1). The immunofluorescence results of paraffin sections (red: COL1A2, blue: nucleus) show that after DIP treatment, the number of colon fibroblasts in child patients is significantly decreased. The right side shows the difference in the number of fibroblasts among the three groups by immunofluorescence on the left side. ****: P<0.0001; and ***: P<0.001.

FIG. 9 shows the PDE1 inhibitor (hereinafter sometimes abbreviated as PDE1i) ITI214 can inhibit fibrosis and effectively relieve colitis symptoms in mice. (A) shows the weight changes of mice. PDE1i significantly increases the weight on day 7. (B) shows the changes of survival rate. PDE1i increases the survival rates of mice with colitis from 50% to 80%. (C) The left side is representative H&E staining pictures of colon tissue, and the right side is pathological score according to H&E statistics. PDE1i significantly restores the structures of colon epithelium and lamina propria, reduces the infiltration of inflammatory cells, and inhibits fibrosis. (D) shows the colon length. PDE1i significantly improves the length of colon with colitis. (E) shows the score en for the disease activity index of colitis. PDE1i significantly reduces the severity of colitis.

FIG. 10 shows a PDE1i inhibitor ITI214 can effectively inhibit proliferation and inflammatory function of fibroblasts. The effects of Dipyridamole and the PDE1 inhibitor ITI-214 on the proliferation and apoptosis of normal intestinal fibroblast CCD-18Co are detected. Cells are continuously stimulated with 20 μM pan PDE inhibitor DIP or 0.5 μM PDE1 inhibitor ITI-214, and the cells at 48 hours are collected respectively for flow analysis, crystal violet staining and protein extraction. Meanwhile, an intestinal interstitial fibrosis model is established in the CCD-18Co cell line, and 20 μM DIP or 0.5 μM PDE1 inhibitor ITI-214 is added one hour in advance. The CCD-18Co cells are continuously stimulated by TGF-β (10 ng/ml) and TNF-α (40 ng/ml), and the cells at 48 hours are collected to extract protein. The protein expression levels of α-SMA, COL1A2 and FAP are detected by Western blot. The proliferation and apoptosis of cells are detected by crystal violet staining and flow analysis of early/late apoptosis. (A) and (C) show that the Dipyridamole inhibits the proliferation of fibroblasts; (B) shows that Dipyridamole promotes the apoptosis of fibroblasts; (D) shows that Dipyridamole inhibits the protein expression levels of α-SMA, COL1A2 and FAP in cells; (E) shows that ITI-214 promotes apoptosis of fibroblasts; and (F) shows that ITI-214 inhibits the protein expression levels of α-SMA, COL1A2 and FAP in cells. The above experimental results show that the pan PDE inhibitor Dipyridamole and the PDE1 inhibitor ITI214 can effectively inhibit the proliferation of fibroblasts, promote the apoptosis of fibroblasts, and inhibit the progress of fibrosis. The above experimental results show that the PDE1i inhibitor ITI214 can effectively inhibit proliferation and inflammatory function of fibroblasts.

FIG. 11 shows the PDE2 inhibitor (hereinafter sometimes abbreviated as PDE2i) PF-05085727 can inhibit fibrosis and effectively relieve colitis symptoms in mice. (A) shows the weight changes of mice. (B) shows the survival rate. PDE2i increases the survival rate of the disease from 40% to 100% (coincided with the control group). (C) The left side is a representative H&E staining picture of colon tissue of each group, and the right side is pathological score of each group. It is found that, in comparison to the model group, the colonic tissue structure is significantly restored, the epithelial structure is more complete, the fibrosis is reduced, and inflammatory cells are reduced. (D) shows the colon length. PDE2i significantly improves the colon length of mice with colitis. (E) shows the disease activity index score. PDE2i does not aggravate the activity degree of the disease.

FIG. 12 shows the PDE9 inhibitor (hereinafter sometimes abbreviated as PDE9i) PF-04447943 can inhibit fibrosis and effectively relieve colitis symptoms in mice. (A) shows the weight changes. PDE9i significantly inhibits the weight loss of colitis from day 6. (B) shows the survival rate. PDE9i increases the survival rate of colitis to 100% (coincided with the control group). (C) The left side is typical H&E staining pictures of colon, and the right side is pathological score. It is found that, in comparison to the model group, PDE9i can significantly restore the colonic tissue structure, the epithelial structure is more complete, the infiltration of inflammatory cells is decreased, and the fibrosis is reduced. (D) shows the colon length. PDE9i can significantly improve the colon length of mice with colitis. (E) shows the disease activity index score of mice. PDE9i significantly inhibits experimental colitis from day 6.

FIG. 13 shows the PDE3 inhibitors (hereinafter sometimes abbreviated as PDE3i) Milirinone, Cilostazol and Vesnarinone can inhibit fibrosis and effectively relieve colitis. (A) shows the weight changes. The weight does not decrease significantly. (B) shows typical H&E staining pictures of colon, and it is found that, in comparison to the model group, the PDE3 inhibitor group can significantly restore the colonic tissue structure, the epithelial structure is more complete, the infiltration of inflammatory cells is decreased, and the fibrosis is reduced. The above experimental results show that the PDE3 inhibitor can effectively improve symptoms of DSS-induced mouse colitis.

FIG. 14 shows the PDE4 inhibitors (hereinafter sometimes abbreviated as PDE4i) Roflumilast can inhibit fibrosis and effectively relieve colitis. (A) shows the weight changes. PDE4i does not decrease the weight significantly. (B) shows typical H&E staining pictures of colon, and it is found that, in comparison to the model group, the PDE4i group can significantly restore the colonic tissue structure, the epithelial structure is more complete, the infiltration of inflammatory cells is decreased, and the fibrosis is reduced. The above experimental results show that the PDE4 inhibitor Roflumilast can effectively relieve symptoms of DSS-induced mouse colitis.

FIG. 15 shows the PDE5 inhibitors (hereinafter sometimes abbreviated as PDE5i) Icariin can inhibit fibrosis and effectively relieve colitis. (A) shows the weight changes. PDE5i significantly inhibits the weight loss of colitis from day 6. (B) shows typical H&E staining pictures of colon, and it is found that, in comparison to the model group, the PDE5i group can significantly restore the colonic tissue structure, the epithelial structure is more complete, the infiltration of inflammatory cells is decreased, and the fibrosis is reduced.

FIG. 16 shows the PDE10 inhibitors (hereinafter sometimes abbreviated as PDE10i) Mardepodect hydrochloride can inhibit fibrosis and effectively relieve colitis. (A) shows the weight changes. PDE10i does not decrease the weight significantly. (B) shows typical H&E staining pictures of colon, and it is found that, in comparison to the model group, the PDE10i group can significantly restore the colonic tissue structure, the epithelial structure is more complete, the infiltration of inflammatory cells is decreased, and the fibrosis is reduced. The above experimental results show that the PDE10 inhibitor Mardepodect hydrochloride can effectively alleviate symptoms of DSS-induced mouse colitis.

### DETAILED DESCRIPTION

The fibrosis described in the present disclosure has a known meaning in the art, and is often shown as increase of fibrillar connective tissues in organ tissues and decrease of parenchymal cells. The meaning of the fibrosis covers fibrosis of various tissues and organs, comprising, but not limited to, liver, gallbladder, lung, kidney, bladder, heart, blood vessel, eye, skin, pancreas, gastrointestinal, bone marrow, penis, breast, and muscle.

For example,

Fibrosis of liver and gallbladder comprises, for example, cirrhosis, hepatic fibrosis, liver injury, hepatic failure, biliary atresia.

Pulmonary fibrosis comprises, for example, idiopathic pulmonary fibrosis (IPF), silicosis, cystic fibrosis (CF), pulmonary hypertension (PH), and related diseases.

Gastrointestine fibrosis comprises fibrosis involved in gastrointestinal tract diseases, for example, colitis, undifferentiated (also known as indeterminate) colitis, Crohn's disease, ulcerative colitis, chronic colitis, chronic eosinophilic colitis and fibrosis in other inflammatory bowel diseases. The gastrointestinal tracts in the present disclosure comprise stomach, duodenum, small intestine, colon and other gastrointestinal tracts.

Muscle fibrosis comprises, for example, muscular dystrophy.

Kidney fibrosis comprises, for example, tubulointerstitial fibrosis (TIF), renal interstitial fibrosis.

Those skilled in the art should understand that the fibrosis of the present disclosure also comprises fibrotic tumors and cancers.

Liver and Gallbladder

With regard to fibrosis of liver and gallbladder, in-depth researches and experiments on the pathogenesis of biliary atresia, dominant cells causing this immune response, and the molecular mechanism thereof by the inventors show that PDE inhibitor can relieve inhibition of cAMP pathway caused by biliary atresia, play an important role in delaying the liver injury and fibrosis, inhibit occurrence and development of inflammation, and inhibit replication of viruses in the liver organ, thus effectively improving the injury caused by biliary atresia.

Specifically, PDE is widely expressed in various immune cell subtypes in liver tissues of patients with biliary atresia. This provides an effect target for the use of the PDE inhibitor in the biliary atresia diseases. In cell experiments in vitro, after stimulation by using the PDE inhibitor such as Dipyridamole, it is found that the fibroblast-related genes expression levels of a hepatic stellate cell line is significantly reduced Administration of Dipyridamole in a RRV-induced biliary atresia animal model can protect the virus-infected mice from developing into biliary atresia, without jaundice, and the liver injury and fibrosis degree are reduced. It is worth noting that the expression level of NSP3 molecule reflecting virus replication in vivo is significantly reduced in livers of the mice treated with the PDE inhibitor such as Dipyridamole, which indicates that the PDE inhibitor protects the livers from virus invasion and virulence. In addition, after administration of the PDE inhibitor, infiltration of inflammatory cells and expression of inflammatory factors in the livers of the mice with biliary atresia induced by the RRV virus are both significantly reduced.

The above brand new discovery of the present disclosure (that the immune cells of the livers of the patients with biliary atresia express a variety of PDEs) brings a new therapeutic method of applying the PDE inhibitor to treat biliary atresia, and also brings new medical use of the PDE inhibitor, which may be used for treating biliary atresia.

In the absence of specific therapies and in the case of high mortality, it is of great scientific significance and clinical value to delay the liver injury caused by biliary atresia, improve the living condition and treat biliary atresia through the PDE inhibitor such as Dipyridamole.

Gastrointestinal Tract

With regard to fibrosis of gastrointestinal tract, it is found by the inventors that the PDE inhibitor such as Dipyridamole relieves proliferation of colitis fibroblasts in mice and clinical experiments. The results show that after DIP injection, the number of fibroblasts in colon of mice with chronic colitis is decreased significantly; and by treatment with the PDE inhibitor such as Dipyridamole, the number of fibroblasts in colon of child patients is significantly reduced as well. In this way, it is indicated that the PDE inhibitor such as dpyridamole can effectively treat gastrointestinal tract fibrosis.

In addition, it is also found by the inventors through experiments that inhibitors such as PDE1, PDE4 and PDE8 may prevent and treat weight loss of mice with chronic colitis. For example, the PDE1 inhibitor ITI214 can effectively relieve the weight loss of mice, improve colitis symptoms of mice, inhibit the fibrosis, and treat enteritis, thus greatly improving the survival rate. The PDE2 inhibitor PF-05085727 can effectively relieve the colitis symptoms of mice, significantly restore the colon tissue structure, make the epithelial structure more complete, and reduce the fibrosis, thus improving the survival rate. The PDE9 inhibitor PF-04447943 can effectively relieve the colitis symptoms of mice, significantly improve the weight loss of mice, significantly restore the colon tissue structure, make the epithelial structure more complete, and reduce the fibrosis, thus improving the survival rate. The PDE3 inhibitors Milirinone, Cilostazol and Vesnarinone can inhibit the fibrosis, and improve the colitis; compared with the model group, the colonic tissue structure is significantly restored, the epithelial structure is more complete, infiltration of inflammatory cells is reduced, and the fibrosis is reduced. The PDE4 inhibitor Roflumilast can inhibit the fibrosis, and improve the colitis; compared with the model group, the colonic tissue structure is significantly restored, the epithelial structure is more complete, infiltration of inflammatory cells is reduced, and the fibrosis is reduced. The PDE5 inhibitor Icariin can inhibit the fibrosis, and improve the colitis; compared with the model group, the colonic tissue structure is significantly restored, the epithelial structure is more complete, infiltration of inflammatory cells is reduced, and the fibrosis is reduced. The PDE10 inhibitor Mardepodect hydrochloride can inhibit the fibrosis, and improve the colitis; compared with the model group, the colonic tissue structure is significantly restored, the epithelial structure is more complete, infiltration of inflammatory cells is reduced, and the fibrosis is reduced.

Lung

With regard to fibrosis of lung, it is found by the inventors that, in an in vitro cell test of SeV virus infection, addition of the PDE inhibitor such as Dipyridamole significantly promotes mRNA and protein levels of IFN-β in cells; in the mouse model with RNA virus VSV infection, it is shown that the infiltration of inflammatory cells in the lungs of mice treated with Dipyridamole is significantly reduced, and the alveolar injury is alleviated. These results all indicate that PDE inhibitors such as Dipyridamole are effective in preventing and treating pulmonary inflammation, immunomodulation and preventing and treating fibrosis. It is also found by the inventors in the experiments that the inhibitors PDE3 and PDE5 can significantly promote the mRNA and protein levels of IFN-β in cells.

It was found by the inventors that Dipyridamole, as a pan PDE inhibitor, has a good effect of inhibiting fibrosis in many organs and tissues such as liver, gallbladder, gastrointestinal tract, and lung, and can resist inflammation and improve immune environment. This provides an important idea for use of the pan PDE inhibitor, and prompts a role of the inhibitor in various fibrotic diseases.

In the present disclosure, patients or subjects with fibrotic diseases are not limited by age or sex, and may be children, adults and the elderly, wherein the children may range from, for example, newborns to children of 12 years old, and infants of 1 year old to children of 6 years old. The drug for treating fibrotic diseases in the present disclosure may also be used for treating other mammals, such as monkey, cattle, horse, pig, mouse, rat, hamster, rabbit, cat, dog, sheep, and goat.

The treatment in the present disclosure also comprises prevention, for example, a patient who is expected to have a high risk of disease due to some factors related to the disease but has not suffered from the disease; or a patient who has suffered from the disease but has no self-conscious symptom, is administrated with the drug of the present disclosure; or a patient who is afraid of recurrence of the disease after treatment is administrated with the drug of the present disclosure.

The phosphodiesterase described in the present disclosure has a known meaning in the art. It is known in the art that the phosphodiesterase has functions of hydrolyzing second messengers (cAMP, cyclic adenosine monophosphate or cGMP, and cyclic guanosine monophosphate) in cells, and degrading the cAMP or the cGMP in cells, thus terminating biochemical effects conducted by these second messengers.

The meaning of the phosphodiesterase inhibitor in the present disclosure is known in the art. It is known in the art that the phosphodiesterase family comprises PDE1, PDE2, PDE3, PDE4, PDE5, PDE6, PDE7, PDE8, PDE9, PDE10, PDE11, etc., each of which has a variety of isozyme subtypes, for example, PDE4 comprises subtypes such as PDE4A, 4B, 4C and 4D.

The phosphodiesterase inhibitor of the present disclosure comprises a drug inhibiting any one or more from the phosphodiesterase family, comprising a selective or non-selective phosphodiesterase inhibitor. The inhibitor comprises, but is not limited to a PDE1 inhibitor, a PDE2 inhibitor, a PDE3 inhibitor, a PDE4 inhibitor, a PDE5 inhibitor, a PDE6 inhibitor, a PDE7 inhibitor, a PDE8 inhibitor, a PDE9 inhibitor, a PDE10 inhibitor, a PDE11 inhibitor, or an inhibitor with an inhibitory effect on various members from the family and a drug with an inhibitory effect on other members from the phosphodiesterase family. An inhibitor with an inhibitory effect on PDE1, and/or PDE2, and/or PDE3, and/or PDE4, and/or PDE5, and/or PDE8, and/or PDE9, and/or PDE10 is preferred. According to the present disclosure, it is known that an inhibitor with inhibitory activities on multiple PDE subtypes, which is sometimes called a pan PDE inhibitor and also called a non-specific PDE inhibitor, such as Dipyridamole, is effective to multiple subtypes such as PDE5, PDE3, PDE4, and PDE2.

Specifically, the phosphodiesterase inhibitor of the present disclosure is not particularly limited as long as having an inhibitory effect on phosphodiesterase. It is known that the inhibitor comprises Nimodipine, Vinpocetine, IC86340, IC224, EHNA, BAY60-7750, IC933, Dipyridamole, Cilostazol, Cilostamide, Milrinone, Amrinone, Enoximone, Siguazodan, Theophylline, Rolipram, Piclamilast, Roflumilast, Cilomilast, Apremilast, Sildenafil, Vardenafil, Tadanafil, Zaprinast, Udinafil, BRL-50481, TI214, PF-05085727, PF-04447943, Milirinone, Cilostazol, Vesnarinone, Roflumilast, Icariin and Mardepodect hydrochloride, IC242, and quinazoline-class and thiadiazole-class small molecule compounds S14 and VP1.15 discovered based on computer simulation and other drugs inhibiting PDE, etc. The PDE3 inhibitor comprises Milrinone, Amrinone, and Cilostazol, etc. The PDE7 inhibitor comprises IC242 and BRL50481, etc. The PDE6 inhibitor comprises Sidenafil, etc. The PDE4 inhibitor comprises Cilomilast and Rolipram, etc. The PDE12 inhibitor comprises PDE12-IN-3, etc. The PDE pan-inhibitor comprises Theophylline, Dipyridamole (sometimes referred to as Dip), and Rottlerin.

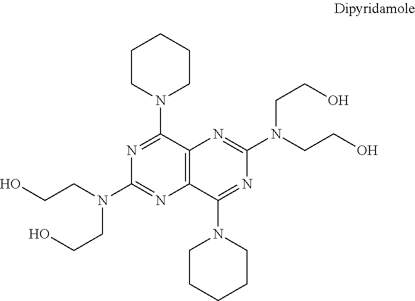 [SMILES: OCCN(CCO)c1nc(N2CCCCC2)c2nc(N(CCO)CCO)nc(N3CCCCC3)c2n1]

Those skilled in the art may understand that forms of an active ingredient in a drug for treating various fibrotic diseases described in the present disclosure are not limited, and may be various forms, comprising an active compound itself, a free state, a salt, an ester, an isomer, an optical isomer, a stereoisomer, a regional isomer, a geometric isomer, a hydrate or an anhydrate, a solvate or a non-solvate, an amorphous form, a crystal, a pharmaceutical co-crystal or co-crystal salt, a derivative, a prodrug, etc. The prodrug comprises a compound which can be converted into the active ingredient due to reactions of enzyme, gastric acid, etc. under a physiological condition in an organism, which is namely a compound capable of being converted into the active ingredient by enzymatic oxidation, reduction, hydrolysis, etc.; and a compound capable of being converted into the active ingredient by hydrolysis due to the gastric acid, and so on. The co-crystal or co-crystal salt refers to a crystalline substance composed of two or more specific substances. At room temperature, each substance is a solid and has different physical properties (such as structure, melting point, fusing heat, hygroscopicity, solubility, stability). The co-crystal or co-crystal salt may be prepared by a known cocrystallization method.

For example, forms of active ingredients of various phosphodiesterase inhibitors (such as Dipyridamole, ITI214, PF-05085727, PF-04447943, Milirinone, Cilostazol, Vesnarinone, Roflumilast, Icariin and Mardepodect hydrochloride) in the present disclosure are not limited, and the forms comprise an active compound itself, a free state, a salt, an ester, an isomer, an optical isomer, a stereoisomer, a regional isomer, a geometric isomer, a hydrate, a non-hydrate, a solvate or a non-solvate, an amorphous form, a crystal, a pharmaceutical co-crystal or co-crystal salt, a derivative, and a prodrug, etc.

In the present disclosure, when the active ingredient is mentioned, it is intended to cover the above various forms of the active ingredient, for example, when Dipyridamole, ITI214, PF-05085727, PF-04447943, etc. are mentioned, it is intended to cover the above various forms of Dipyridamole, ITI214, PF-05085727, PF-04447943, Milirinone, Cilostazol, Vesnarinone, Roflumilast, Icariin, and Mardepodect hydrochloride, comprising but being not limited to a free form, an ester, a salt, a derivative, a prodrug and other modified forms.

In the present disclosure, when the active ingredient is the salt, examples of the salt comprise a metal salt, an ammonium salt, a salt formed with an organic base, a salt formed with an inorganic acid, a salt formed with an organic acid, and a salt formed with a basic or acidic amino acid. Preferred examples of the metal salt comprise an alkali metal salt, such as sodium salt and potassium salt; an alkaline-earth metal salt, such as a calcium salt, a magnesium salt and a barium salt; and an aluminum salt. Preferred examples of the salt formed with the organic base comprise salts formed with the following organic bases: trimethylamine, triethylamine, pyridine, methylpyridine, 2,6-dimethylpyridine, ethanolamine, diethanolamine, triethanolamine, cyclohexylamine, dicyclohexylamine, and N,N′-dibenzylethylenediamine. Preferred examples of the salt formed with the inorganic acid comprise salts formed with hydrochloric acid, hydrobromic acid, nitric acid, sulfuric acid, and phosphoric acid. Preferred examples of the salt formed with the organic acid comprise salts formed with the following organic acids: formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, and p-toluenesulfonic acid. Preferred examples of the salt formed with the basic amino acid comprise salts formed with the following basic amino acids: arginine, lysine and ornithine. Preferred examples of the salt formed with the acidic amino acid comprise salts formed with the following acidic amino acids: aspartic acid and glutamic acid.

A pharmaceutically acceptable salt is preferred. For example, when the active ingredient contains an acidic functional group, an example thereof comprises the inorganic salt, such as the alkali metal salt (such as a sodium salt and a potassium salt), the alkaline earth metal salt (such as a calcium salt and a magnesium salt), and the ammonium salt. When the compound contains a basic functional group, an example thereof comprises the salt formed with the inorganic acid, such as hydrochloric acid, hydrobromic acid, nitric acid, sulfuric acid and phosphoric acid; and the salt formed with the organic acid, such as acetic acid, phthalic acid, a fumaric acid, oxalic acid, tartaric acid, maleic acid, citric acid, succinic acid, methanesulfonic acid, benzenesulfonic acid, and p-toluenesulfonic acid.

For example, the phosphodiesterase inhibitor of the present disclosure may be in modified forms such as a free state, a salt, an ester, and various other derivatives and prodrugs. For example, Dipyridamole may be in modified forms such as a free state, an ester, a salt, a derivative, and a prodrug.

The PDE inhibitor for fibrotic diseases described in the present disclosure may be administered in a form of an active compound itself or a mixture of the active compound and a pharmaceutically acceptable carrier.

Various organic or inorganic carrier substances commonly used as raw materials of preparations may be used as the pharmaceutically acceptable carrier, without particular limitation, which may be an excipient, a lubricant, an adhesive and a disintegrant in a solid preparation; a vehicle, a solubilizer, a suspending agent, an isotonic agent, a buffer and an analgesic in a liquid preparation. In addition, preparation additives comprising a preservative, an antioxidant, a stabilizer, a colorant, and a sweetener may also be used as needed.

A preparation form of the PDE inhibitor of the present disclosure for fibrotic diseases is not particularly limited, and may be used as a drug for non-oral administration or oral administration, such as a form of coating with liposome or exosome. The drug of the present disclosure may be any one of solid preparations such as powders, granules, tablets or capsules, or liquid preparations such as syrups or emulsions. The drug for treating fibrotic diseases, such as gastrointestinal tract disease, may be safely administered in the following forms (such as intravenous administration, intramuscular administration, subcutaneous administration, intraorgan administration, nasal administration, intradermal administration, drop, intracerebral administration, intrarectal administration, vagina administration, intraperitoneal administration, intratumor administration, tumor proximal end administration, and lesion administration): tablets (comprising sugar-coated tablets, film-coated tablets, sublingual tablets, orally disintegrating tablets, and buccal tablets, etc.), pills, powders, granules, capsules (comprising soft capsules and microcapsules), lozenges, syrups, liquids, emulsions, suspensions, controlled-release preparations (such as immediate-release preparations, sustained-release preparations and sustained-release microcapsules), aerosols, films (such as orally disintegrating films and oral mucosa adhesive films), injections (such as subcutaneous injections, intravenous injections, intramuscular injections, and intraperitoneal injections), intravenous infusions, transdermal absorption preparations, creams, ointments, lotions, adhesive preparations, suppositories (such as rectal suppositorys and vaginal suppositorys), medicinal granules, nasal preparations, lung preparations (such as inhalants), and eye drops.

The content of PDE inhibitor of the present disclosure in a pharmaceutical composition is changed based on the dosage form and the dosage of the compound. For example, the content is in a range of about 0.1 wt % to 100 wt %.

The dosage of the PDE inhibitor for fibrotic diseases in the present disclosure is not particularly limited as long as being a therapeutically effective dosage. In the description, the term “therapeutically effective dosage” refers to a dosage bringing a therapeutic effect to a subject, for example, for the subject who is administered with this dosage of drug, symptom or state of the disease is relieved, alleviated or eliminated, or development of symptom or state of the disease is delayed or inhibited, compared with the subject who is not administered with this dosage of drug. The therapeutically effective dosage may be appropriately determined by a doctor according to age, weight, sex and symptom severity of the subject. For example, children are administered once a day or multiple times a day, with a dosage of 0.1 mg/kg/day to 100 mg/kg/day, 1 mg/kg/day to 50 mg/kg/day, and 3 mg/kg/day to 20 mg/kg/day.

The PDE inhibitor for treating fibrotic diseases may be combined with other drugs. The other drugs comprise: an anti-atherosclerosis drug, an anti-thrombosis drug, an anti-heart failure drug, an anti-arrhythmia drug, an anti-hypertension drug, a drug for treating diabetes, a drug for treating diabetic complications, a drug for increasing HDL, an anti-hyperlipidemia drug, an anti-obesity drug, a diuretic, an anti-inflammatory agent, an anti-gout drug, a chemotherapy agent, an immunotherapy agent such as an anti-TNFα alpha drug, a hormone drug such as a glucocorticoid drug, a drug for treating osteoporosis, an anti-dementia drug, a drug for improving erectile dysfunction, a drug for treating urinary incontinence, and a drug for treating dysuria. Other drugs may be a low molecular compound or a high molecular protein, polypeptide, antibody, and vaccine, etc.

There is no limitation on the administration time of PDE inhibitor and other drugs for treating fibrotic diseases of the present disclosure and they may be administered to a patient simultaneously or in a staggered manner. The dosage of the other drugs may be appropriately determined based on the dosage used in clinical conditions, and may be appropriately determined according to the patient to be administered, the administration way, the targeted disease, the symptom, and the combined drug.

The PDE inhibitor for fibrotic diseases of the present disclosure may be formed into the combined drug with the above other pharmaceutical active ingredients. The combined drug may be formed into a single preparation with active ingredients in the same preparation, or multiple preparations with active ingredients in different preparations.

The present disclosure is preferable in the following aspects.

The present disclosure preferably uses Dipyridamole for preventing and/or treating a fibrotic disease, and pharmaceutical use thereof.

The present disclosure preferably uses a pan PDE inhibitor for preventing and/or treating a fibrotic disease, and pharmaceutical use thereof.

The present disclosure preferably uses PDE1, PDE2, PDE3, PDE4, PDE5, PDE8, PDE9 and PDE10 inhibitors for preventing and/or treating a fibrotic disease, and pharmaceutical use thereof.

The present disclosure preferably uses Dipyridamole for preventing and/or treating a fibrotic disease of liver or gallbladder, and pharmaceutical use thereof.

The present disclosure preferably uses Dipyridamole for preventing and/or treating biliary atresia, cirrhosis, chronic liver injury, hepatic failure and hepatic fibrotic diseases, and pharmaceutical use thereof.

The present disclosure preferably uses a pan PDE inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE1 inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE2 inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE3 inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE4 inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE4B inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE5 inhibitor for preventing and/or treating a fibrotic disease of liver or gallbladder, especially biliary atresia, cirrhosis, chronic liver injury, hepatic failure and hepatic fibrotic diseases, and pharmaceutical use thereof.

The present disclosure preferably uses a PDE8 inhibitor for preventing and/or treating a fibrotic disease of liver or gallbladder, especially biliary atresia, cirrhosis, chronic liver injury, hepatic failure and hepatic fibrotic diseases, and pharmaceutical use thereof. The present disclosure preferably uses PDE3B, PDE7A, PDE6D, PDE4D, PDE4B and PDE12 inhibitors for preventing and/or treating a fibrotic disease of liver or gallbladder, especially biliary atresia.

The present disclosure preferably uses Dipyridamole for preventing and/or treating a gastrointestinal tract fibrotic disease, and pharmaceutical use thereof.

The present disclosure preferably uses Dipyridamole for preventing and/or treating fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a pan PDE inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE1 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE1 inhibitor ITI214 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE2 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE2 inhibitor PF-05085727 for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof. The PDE2 inhibitor, such as PF-05085727, can significantly restore the structure of colon tissue, and make the epithelial structure intact and reduce fibrosis.

The present disclosure preferably uses a PDE3 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses PDE3 inhibitors Milirinone, Cilostazol and Vesnarinone for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof. The PDE3 inhibitor, such as Milirinone, Cilostazol and Vesnarinone, can significantly restore the structure of colon tissue, make the epithelial structure intact and reduce fibrosis.

The present disclosure preferably uses a PDE4 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE4 inhibitor Roflumilast for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof. The PDE4 inhibitor, such as Roflumilast, can significantly restore the structure of colon tissue, and make the epithelial structure intact and reduce fibrosis.

The present disclosure preferably uses a PDE4B inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE5 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE5 inhibitor Icariin for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof. The PDE5 inhibitor, such as Icariin, can significantly restore the structure of colon tissue, make the epithelial structure intact and reduce fibrosis.

The present disclosure preferably uses a PDE8 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE9 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE9 inhibitor PF-04447943 for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof. The PDE9 inhibitor, such as PF-04447943, can significantly restore the structure of colon tissue, make the epithelial structure intact and reduce fibrosis.

The present disclosure preferably uses a PDE10 inhibitor for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof.

The present disclosure preferably uses a PDE10 inhibitor Mardepodect hydrochloride for preventing and/or treating a gastrointestinal tract fibrotic disease, especially fibrosis of stomach, duodenum, small intestine or colon, for example, colitis, undifferentiated colitis, Crohn's disease, ulcerative colitis, chronic colitis and chronic eosinophilic colitis, and pharmaceutical use thereof. The PDE10 inhibitor, such as Mardepodect hydrochloride, can significantly restore the structure of colon tissue, and make the epithelial structure intact and reduce fibrosis.

The present disclosure preferably uses Dipyridamole for preventing and/or treating a pulmonary fibrotic disease, and pharmaceutical use thereof.

The present disclosure preferably uses Dipyridamole for preventing and/or treating an idiopathic pulmonary fibrotic disease, silicosis or pulmonary hypertension, and pharmaceutical use thereof.

The present disclosure preferably uses a pan PDE inhibitor for preventing and/or treating a pulmonary fibrotic disease, especially idiopathic pulmonary fibrotic disease, silicosis or pulmonary hypertension, and pharmaceutical use thereof.

The present disclosure preferably uses a PDE1 inhibitor for preventing and/or treating a pulmonary fibrotic disease, especially idiopathic pulmonary fibrotic disease, silicosis or pulmonary hypertension, and pharmaceutical use thereof.

The present disclosure preferably uses a PDE3 inhibitor for preventing and/or treating a pulmonary fibrotic disease, especially idiopathic pulmonary fibrotic disease, silicosis or pulmonary hypertension, and pharmaceutical use thereof.

The present disclosure preferably uses a PDE4 inhibitor for preventing and/or treating a pulmonary fibrotic disease, especially idiopathic pulmonary fibrotic disease, silicosis or pulmonary hypertension, and pharmaceutical use thereof.

The present disclosure preferably uses a PDE5 inhibitor for preventing and/or treating a pulmonary fibrotic disease, especially idiopathic pulmonary fibrotic disease, silicosis or pulmonary hypertension, and pharmaceutical use thereof.

The present disclosure preferably uses a PDE8 inhibitor for preventing and/or treating a pulmonary fibrotic disease, especially idiopathic pulmonary fibrotic disease, silicosis or pulmonary hypertension, and pharmaceutical use thereof.

### EXAMPLES

The present disclosure will be further explained in detail with reference to the following examples, but the present disclosure is not limited thereto.

### Example 1: Single-Cell Sequencing and Bioinformatics Analysis of Cells in Liver Tissues and Intestinal Tissues of Children with Biliary Atresia

In order to study occurrence mechanism of autoimmune liver injury caused by biliary atresia, and explore dominant cells and molecular mechanisms that caused this immune response, the present disclosure sorted immune cells (CD45⁺) in the liver tissues of children with BA, and then performed single-cell sequencing and bioinformatics analysis.

1. Collection of clinical specimens and data:

Medical histories of children with biliary atresia and control group, especially laboratory test indexes related to liver injury, were obtained from an electronic medical file system of a hospital. Ethical statement: this project was approved by the Medical Ethics Committee of Guangzhou Women and Children Medical Center. The experiments were carried out according to the “International Ethical Standards for Medical Research Involving Human Subjects” stated in the “Declaration of Helsinki”. Informed consents for the project research were obtained from patients or legal guardians of patients. Selection criteria of subjects: the subjects of this study were recruited from the children who underwent hepatobiliary surgery in the Hepatobiliary Surgery Department of Guangzhou Women and Children Medical Center.

Control child patients and child patients with BA were judged according to the following criterias:

(1) Control: child patients of 0-4 months old with hepatobiliary diseases (such as choledochal cyst, liver tumor and cholestasis) needing surgical treatment.

(2) Bbiliary atresia: Child patients of 0-4 months old with obstructive jaundice definitively diagnosed with BA by intraoperative cholangiography and/or liver biopsy.

(3) Exclusion criteria: (1) child patients with systemic inflammatory response syndrome or multiple system malformation; (2) child patients with unclear diagnosis of primary diseases; and (3) child patients whose parents refused to participate in the research or could not get authorization from the parents of the child patients.

2. Flow Cytometry

Firstly, venous blood (2 mL) was extracted for Ficoll density gradient centrifugation, and peripheral blood mononuclear cells (PBMC) were isolated and counted. 0.5 g of liver tissue was ground, filtered and lysed to obtain a single-cell suspension and counted. PBMC and the liver single-cell suspension were washed twice with PBS containing 0.5% BSA and 0.5 mM EDTA and stained with various antibody combinations. The data was obtained on a FACS Aria Sorp system and analyzed by Flowjo 10.4 software Immunostaining were carried out using the following antibody combinations: T cell phenotype and function: CD45, CD3, CD8a, γδT, CD45RA, CD25, CD127, CD44, CD103, CD69, PD-1, CCR2, CCR6, CXCR5 and CXCR3; B cell phenotype: CD45, CD19, CD27, IgD, IgM, IgG, IgA, CD38, CD138, CD10, CD21, CD23, FCRL4 and CD83. To detect the secretion of cytokines, 2×10⁶ cells were resuspended in a medium containing 10% fetal bovine serum, stimulated and cultured in an incubator at 37□ for 4-6 hours with phorbol ester (PMA), Inomycin and Monensin. Cells were collected, broken and fixed, and then stained to detect expression of cytokines IFN-γ, IL-17, IL-10, IL-2 and IL-4, and transcription factors FOXP3, T-bet, RUNX3 and RORγ.

3 Immunofluorescence Staining

Paraffin sections of liver tissues of the control group and BA patients were dewaxed in xylene, added with water, heated to repair exposed antigens, washed with PBS for 3 minutes for 3 times, and sealed with 1% normal goat serum at room temperature for 1 hour. Then the sealing solution was sucked off, and primary antibody was directly added. After being moisturized at 4□ overnight, the reaction system was washed twice with precooled PBS, then incubated with a labeled secondary antibody at room temperature for 1 hour, and avoided from light. The reaction system was washed twice with PBS. Photos were taken under a fluorescence microscope or a laser confocal microscope.

4. Autoantibody Detection by ELISPOT

10 μg/ml of dsDNA, Chromatin, RNP and Ro/SSA autoantibodies were spread on a 96-well filter plate overnight, washed with PBS and sealed with RPMI 1640 medium containing 2% fetal bovine at room temperature for 2 hours. The flow sorted B cells (2,000-10,000) were spread on a plate and incubated in an incubator at 37□ for 18-48 hours, washed with PBS, then added with 1:1,000 IgG secondary antibody and incubated at room temperature for 2 hours. After the reaction, the reaction system was washed with PBS and subjected to color development for about 10 minutes. After the color development was completed, the reaction system was placed in a dark place and dried overnight, and the number of spots was the number of B cells producing autoantibodies.

5. Autoantibody Detection by ELISA

Similar to ELISPOT, 10 μg/ml of dsDNA, Chromatin, RNP and Ro/SSA autoantibodies were spread on a 96-well PVA plate overnight, washed with PBS and sealed with RPMI 1640 medium containing 2% fetal bovine serum at room temperature for 2 hours. Samples (lavage fluid, medium supernatant or plasma) were added into the reaction system and incubated at room temperature for 2 hours, washed with PBS, incubated in the second antibody for 2 hours, and finally subjected to color development for about 20 minutes. After the color development, an absorbance at 405 was measured by a microplate reader.

Results of single-cell sequencing in livers of patients with BA showed that PDE3B, PDE7A, PDE6D, PDE4D, PDE4B and PDE12 were expressed in immune cells of the livers of the patients with BA, wherein PDE4B was the most widely expressed (referring to FIG. 1). This suggested that inhibiting PDE activity could relieve the inhibition of cAMP pathway, and increase the level of cAMP in the liver, thus protecting the liver.

Results of single-cell sequencing in intestinal mucosas of child patients with chronic colitis, Crohn's disease and ulcerative colitis showed that PDE1A and C were specifically expressed in fibroblasts, PDE3A was mainly specifically expressed in epithelial cells, PDE4A was specifically expressed in Lti, PDE4B was specifically expressed in B cells, PDE4C was specifically expressed in epithelial cells, PDE4D was specifically expressed in T&NK cells, PDE5A was specifically expressed in fibroblasts, PDE9A was specifically expressed in epithelial cells, and PDE10A was specifically expressed in fibroblasts.

### Example 2: PDE Inhibitor Dipyridamole Inhibiting Expression of Fibroblast Genes

In vitro cell culture: human hepatic stellate cell lines LX-2 were cultured in vitro and divided into a control group (Nli), a Dipyridamole (4 μM) group, a cytokine TGF-β (5 ng/ml) group and a cytokine TGF-β (5 ng/ml)+Dipyridamole (4 μM) group. The experiment was conducted for 3 days, and cells were collected to extract RNA, which was then reversely transcribed into cDNA, and then qPCR was used to detect the expression of fibroblast genes.

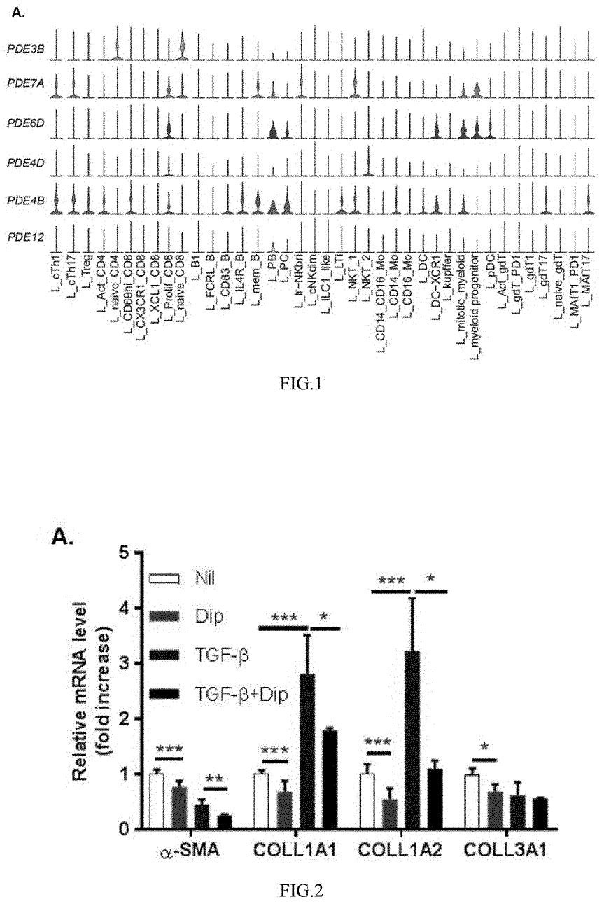

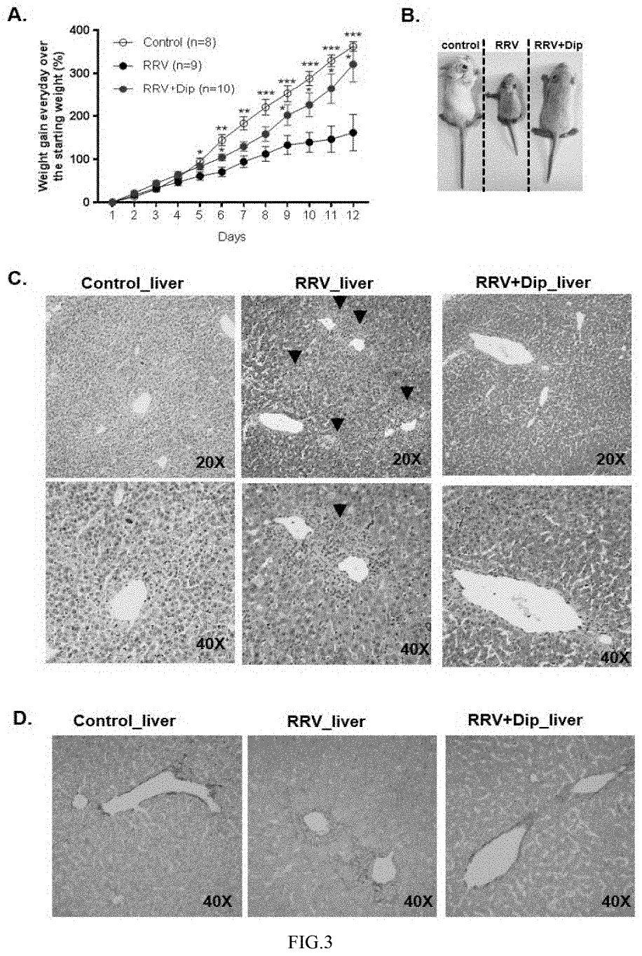

The results showed that: as shown in FIG. 2, Dip significantly inhibited the expression of fibroblast genes α-SMA, COLL1A1, COLL1A2 and COLL3A1. Under the stimulation of the cytokine TGF-β, Dip could still inhibit the expression of the fibroblast genes.

### Example 3: Effect Experiment of PDE Inhibitor Dipyridamole on Biliary Atresia

1. Establishment of Animal Model of Biliary Atresia:

(1) Animals: adult BALB/c pregnant mice, specific pathogen free (SPF), purchased from Guangdong Medical Laboratory Animal Center. The rats were bred in a SPF environment of

Laboratory Animal Center of Guangzhou Medical University. After the pregnant rats gave birth to newborn mice (each pregnant mice gave birth to an average of 8 newborn mice), with an average weight of 1.5 g, the newborn mice were randomly selected for experiments according to experimental groups. This experimental animal treatment method was in conformity with animal ethics standards.

(2) Modeling method: the newborn BALB/c mice were intraperitoneally injected with 20 μL of monkey MMU18006 rotavirus (titer 1.0×10⁶ PFU) within 24 hours after birth to establish BA mouse animal models. For detailed steps, please refer to the earlier published article “Comparison of hepatobiliary injury of different titers of rhesus to newborn mice BALB/c mice, Chinese Journal of Experimental and Clinical Virology, 2017.01.1003-9279”, the contents of which were all cited herein as reference.

(3) Observing the living conditions of mice: comprising the survival rate, growth weight, skin jaundice and changes of liver function.

(4) Detection of immune cells and cytokines immunofluorescence and flow cytometry were used to detect the expression of PD-1⁺T cells and CD21-B cells in liver tissues of this project. For details, please refer to the earlier article “Zhang R. Nanomedicine: nanotechnology, niology, and medicine, 2017, 13 (3): 1041-1050”, all of which were hereby incorporated by reference.

On the established mouse model of acute biliary atresia, Dipyridamole was injected intraperitoneally before the mice were injected with rotavirus (RRV). According to the experimental requirements, the BA models were divided into different experimental groups: 1) control mouse group, 2) BA mouse model group, 3) BA mouse+vehicle group, and 4) BA mouse+Dip group (wherein 50 μg of Dip was injected intraperitoneally per 2 g of weight, supplementary injected on days 3, 6 and 9 after RRV injection, and the experiment was terminated on day 12). The hepatobiliary appearance, jaundice characteristics and survival rate of mice in the above groups were observed, and the number of inflammatory cells infiltration in liver tissues was detected. Cytokines (IL-6, IL-8, IL-10, IL-1b, IL-18, IFN-γ, IL-17, IL-10, TNF-α, TGF-β, bFGF, PDGF and CTGF), antibody subtypes and autoantibody contents which were proved to be closely related to the biliary atresia and fibrosis were detected in mouse liver cell suspensions.

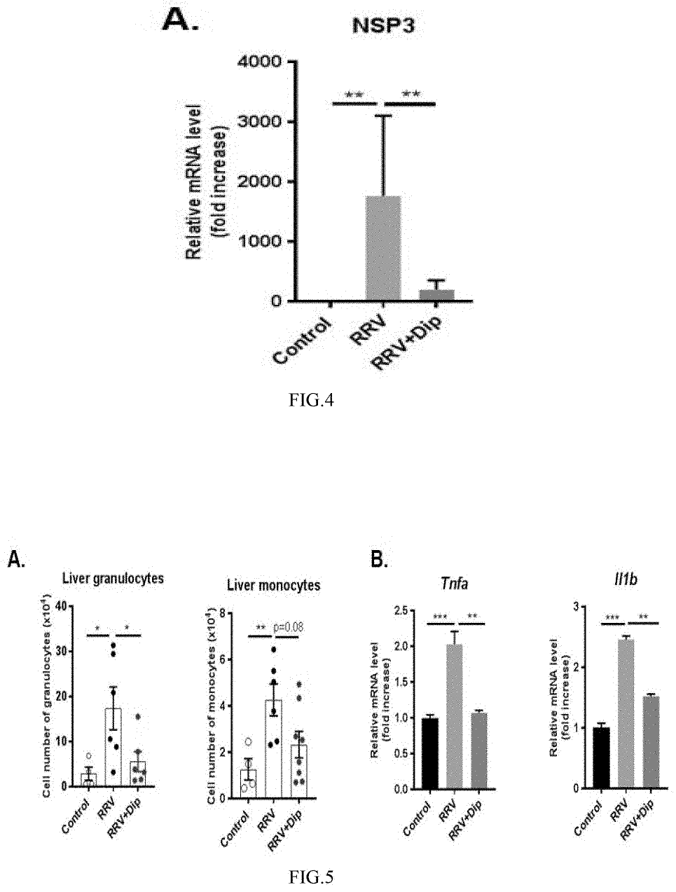

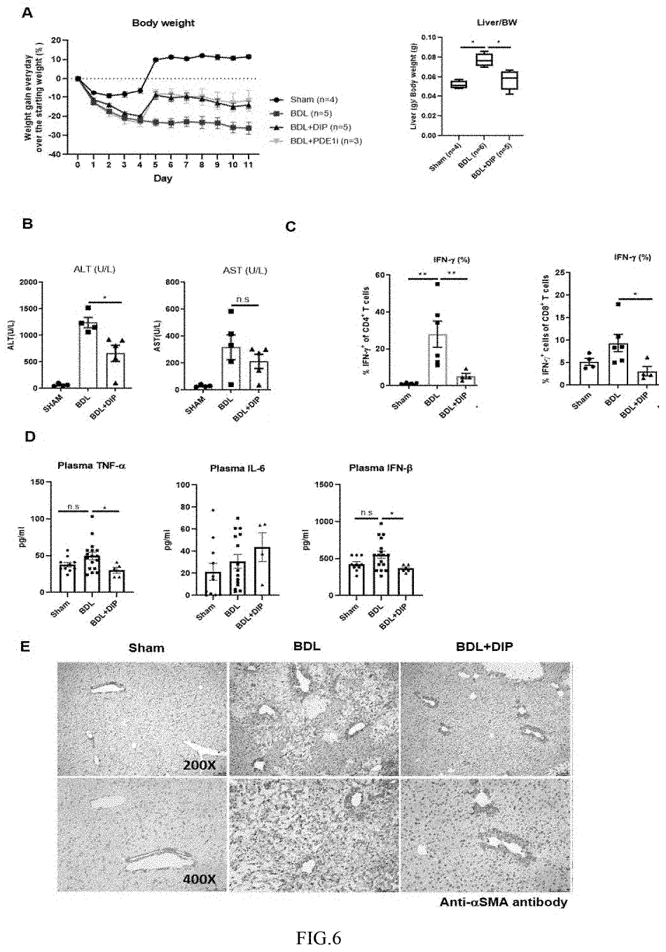

The results showed that: as shown in FIGS. 3-5, using a PDE inhibitor Dipyridamole (Dip) in the model of biliary atresia caused by RRV infection in newborn mice, as shown in FIG. 3, could increase the weights (A) of mice without jaundice (B), suggesting that Dip could prevent the occurrence of RRV-induced biliary atresia; HE staining of liver tissues showed that liver necrosis and infiltration lesions of inflammatory cells decreased in the Dip group (C), while Sirius red staining suggested that liver fiber staining decreased significantly (D). Therefore, the Dip protected liver injury and liver fibrosis caused by RRV virus infection; liver necrosis lesions, infiltration of inflammatory cells and secretion of inflammatory cytokines decreased. FIG. 4 showed that Dipyridamole (Dip) inhibited the replication of RRV virus. Non-structural protein 3 (NSP3) was involved in virus replication in vivo. In FIG. 4(A), Y-coordinate represented the level of NSP3. The qPCR results of mouse liver tissues suggested that the level of NSP3 in the mice of the Dip group was significantly lower than that in the non-drug group, which indicated that Dip could inhibit the invasion and toxicity of virus to the liver and protect the liver. FIG. 5 showed that the infiltration of inflammation-related cells such as neutrophils and monocytes in the livers of RRV mice administered with Dip decreased (A), and the mRNA levels of inflammatory factors TNF-α and IL-1β decreased significantly (B). All these results suggested that Dip could inhibit the liver inflammation caused by RRV virus infection.

### Example 4: Effect Experiment of PDE Inhibitor Dipyridamole on Liver Injury Caused by Bile Duct Ligation (BDL)

Establishment of BDL Model

Male C57/B6J mice aged 6-8 weeks were used for modeling. Before operation, the mice were fasted with water and libitum for 12 hours. The mice were anesthetized by intraperitoneal injection of 10% phenobarbital sodium (100 mg/kg). Depilatory paste was used for abdominal skin shaving. The procedure of bile duct ligation was as follows: after the common bile duct was found through abdominal incision, the common bile duct was cut with 5-0 silk suture, two ends of the incision were both ligated, and the common bile duct was cut between the ligatures. The control group was subjected to sham operation, the common bile duct was exposed, but was not ligated. 4-0 dexon and 2-0 nylon were used for abdominal suture. On the established BDL mouse models, Dipyridamole was injected intraperitoneally, and paired experiments were conducted according to the experimental requirements to divide the BDL models into different experimental groups: 1) sham operation mouse control group, 2) BDL mouse model group, 3) BDL mouse+vehicle group, and 4) BA mouse+Dip group (500 ug of Dip was injected intraperitoneally per 20 g of weight every day). The hepatobiliary appearance, jaundice characteristics and survival rate of mice in the above groups were observed, and the number of inflammatory cells infiltration in liver tissues was detected at the same time. Inflammatory cytokines in the mouse plasma were detected.

The results showed that: as shown in FIG. 6, the models of liver injury caused by bile duct ligation (BDL), administration of DIP to the mice could significantly improve the liver injury caused by BDL, which showed that DIP could slow down a continuous weight loss caused by BDL, and could also significantly reduce the liver weight ratio (A). In addition, the increased liver function index ALT was also significantly reduced in the mice administered with DIP (B). It was found by further analysis that DIP could reduce the level of inflammatory cytokines IFN-γ in liver (C). ELISA results showed that the levels of inflammatory cytokines such as TNFα and IFN-β in plasma were significantly reduced in BDL mice treated with DIP (D). The livers of BDL model were accompanied by the increase of fibroblasts, the destruction of liver tissue structures and the infiltration of inflammatory cells, all of which were significantly improved in mice treated with DIP. Immunohistochemical results suggested that fibroblasts were significantly reduced in BDL mice treated with DIP, the liver tissue structures were clear, and the inflammatory cells were reduced (E). These results all indicated that the PDE inhibitor DIP could inhibit liver inflammation and injury caused by cholestasis and protect the liver.

### Example 5: Effect Experiment of PDE Inhibitor on Pulmonary Fibrosis

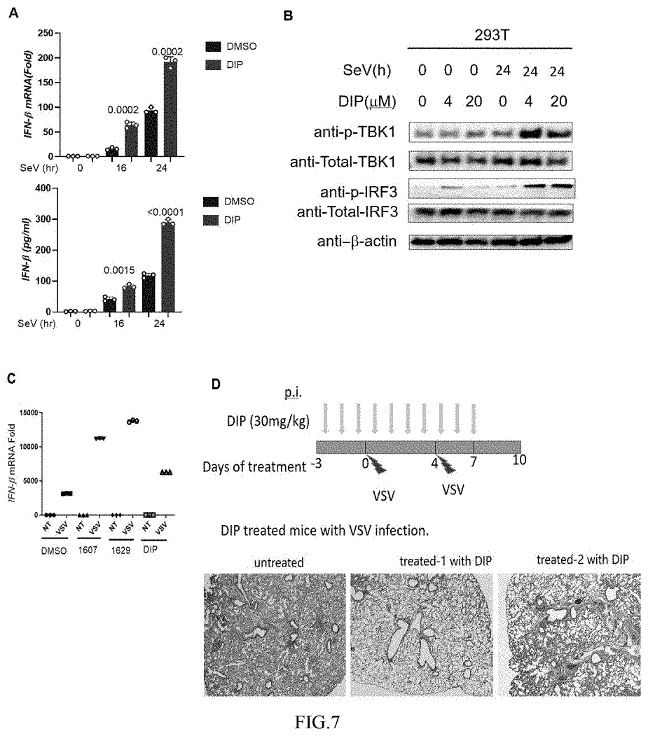

DIP and DMSO were respectively added to lung epithelial cells A549, and the cells were infected with SEV (MOI=1) at the same time, cells and culture supernatants at 0, 16 and 24 hours were collected respectively. Total RNA was extracted from cells, and the mRNA level of IFN-β was detected by fluorescence quantitative PCR. The level of IFN-β in the supernatants was detected by ELISA. Addition of Dip significantly promoted the mRNA and protein levels of IFN-β in cells (referring to FIG. 7A).

293T cells were respectively added with different doses of DIP (0 mM, 4 mM, 20 mM), and infected with SeV (MOI=1). Then the cells were collected, and phosphorylation levels of kinase TBK1 and transcription factor IRF3 were detected by western blot (referring to FIG. 7B).

Different PDE inhibitors (DIP and the like) (5 μM) and DMSO were added to lung epithelial cells A549 respectively, and the cells were infected with SEV (MOI=1) for 24 hours at the same time. The cells were collected and total RNA was collected from the cells, and the mRNA level of IFN-β was detected by fluorescence quantitative PCR. The PDE inhibitor could significantly up-regulate the mRNA level of the type I interferon IFN-β (referring to FIG. 7C).

Mouse models infected by RNA virus VSV were constructed according to the methods known in the art. The mice were injected with Dipyridamole (30 mg/kg) intraperitoneally from day −3 and injected with VSV (10⁸ PFU/g) through tail vein on day 0 and day 4, and the administrating lasted for 7 days. H&E staining of mouse lung showed that the infiltration of pulmonary inflammation in lung and alveolar injury were significantly reduced after DIP treatment (referring to FIG. 7D).

The above results showed the Dipyridamole promoted type I interferon signals and alleviated lung injury caused by virus infection.

### Example 6: Effect Experiment of PDE Inhibitor Dipyridamole on Gastrointestinal Tract Fibrosis

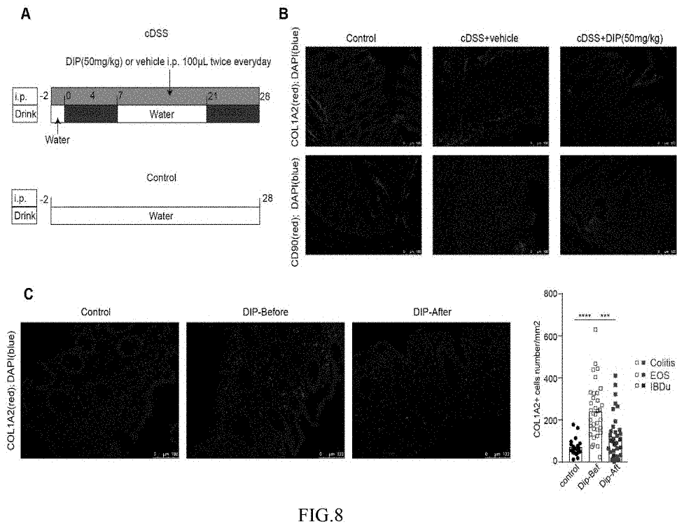

Mice were divided into three groups, and the control group was fed with normal diet and drinking water; the cDSS+DIP (chronic colitis treated with DIP) group was injected intraperitoneally with 100 uL of DIP (50 mg/kg) from day −2, twice a day, which lasted until day 28; and the cDSS+vehicle (chronic colitis control) group was injected intraperitoneally with the same volume of control vehicle from day −2, twice a day, which also lasted until day 28. Meanwhile, in the cDSS+DIP and cDSS+vehicle groups, the normal drinking was replaced with drinking water containing 2% DSS (dextran sulfate sodium) on days 0-7 and days 21-28, and the normal drinking water was provided at the rest of the time. In all groups, on day 28, mice colons were taken for immunofluorescence staining of frozen sections (referring to FIG. 8A).

FIG. 8B showed colon immunofluorescence mentioned above, blue was the nucleus, red (above: COL1A2; below: CD90, all of which were fibroblast indexes). The results prompted that the number of fibroblasts in colon of mice with chronic colitis after DIP injection decreased significantly. Control: N=3; cDSS+DIP (chronic colitis treated with DIP) group: N=3; and cDSS+vehicle (chronic colitis control) group: N=3.

In the preliminary clinical experiment of DIP, the inventors divided child patients into three groups: the control group (normal colon) (N=4), DIP-Before and DIP-after (before and after DIP treatment) (N=7, Colitis was chronic colitis, N=3, EOS was chronic eosinophilic colitis, N=3, and IBDu was undifferentiated inflammatory bowel disease, N=1). The immunofluorescence results of paraffin sections (red: COL1A2, blue: nucleus) showed that the number of colon fibroblasts in child patients after DIP treatment was significantly decreased. The right side showed the difference in the number of fibroblasts among the three groups by immunofluorescence on the left side. ****: P<0.0001; and ***: P<0.001 (referring to FIG. 8C).

The above results showed that Dipyridamole could alleviate the proliferation of colitis fibroblasts in both mice and patients' clinical experiments.

### Example 7: Effect Experiment of PDE1 Inhibitor on Inhibiting Fibrosis and Treating Colitis

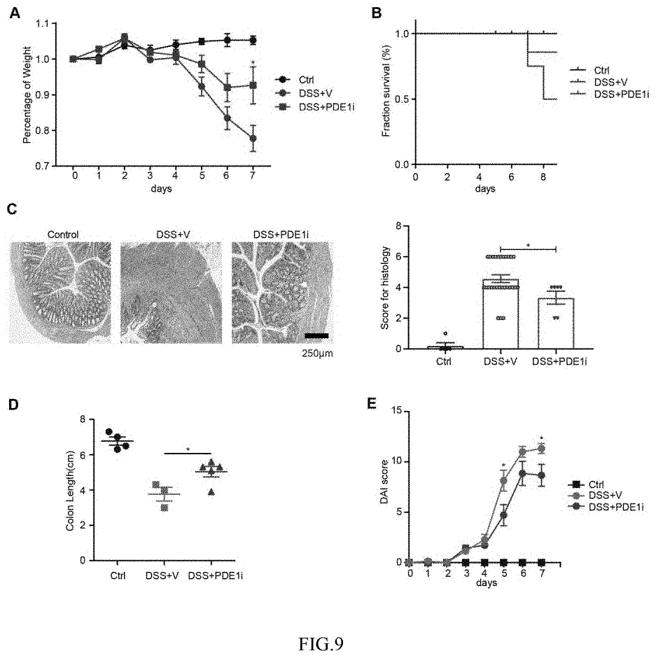

C57BL/6 mice were divided into three groups comprising normal control (Control, n=4), modeling+vehicle control (DSS+V, n=7) and modeling+3 mg/kg PDE1 inhibitor ITI214 (DSS+PDE1i, n=7). ITI214 was purchased from MCE, HY-12501A, CasNo.: 1642303-38-5, and the molecular structure thereof was as follows) for experiment. V represented vehicle, and DSS represented dextran sulfate sodium. The modeling method of the colitis in mice was as follows: on days 0-7, drinking water containing 2% DSS was fed every day, and changed into normal drinking from day 8. The drug and the vehicle were injected intraperitoneally on days 4-8, twice a day, and 100 μL each time. FIG. 9(A) showed the weight changes of mice. PDE1i significantly increased the weight on day 7. FIG. 9(B) showed the survival rate changes. PDE1i increased the survival rates of mice with colitis from 50% to 80%. The left side of FIG. 9(C) was a representative H&E staining picture of colon tissue, and the right side of FIG. 9(C) was a pathological score according to H&E statistics (specific scoring criteria: comprising tissue injury and inflammatory cell infiltration degree in lamina propria. 0 score: no tissue injury or no inflammatory cell infiltration; 1 scores: focal epithelial injury; 2 scores: focal epithelial injury with a small amount of inflammatory cell infiltration in lamina propria; 3 scores: mucosal erosion, ulcer, and partial inflammatory cell infiltration in lamina propria; 4 scores: mucosal erosion, ulcer, and inflammatory cell cluster infiltration in lamina propria; 5 scores: extensive injury in deep intestinal wall, and infiltration of a large number of inflammatory cell in lamina propria; and 6 scores: extensive injury in deep intestinal wall, and infiltration of a large number of inflammatory cell across the wall). The drug significantly restored the structures of colon epithelium and lamina propria, reduces the infiltration of inflammatory cells, and inhibits fibrosis. FIG. 9(D) was the colon length. PDE1i significantly improved the colon length of colitis. FIG. 9(E) showed the disease activity index score en (DAI score) of colitis (specific scoring criteria: 0 score when the weight was lost by 0-1%, 1 score when the weight was lost by 1-5%, 2 score when the weight was lost by 5-10%, 3 scores when the weight was lost by 10-15%, and 4 scores the weight was lost by more than 15%; 0 score when the stool property was normal, 1 score when the stool property was lightly loose, 2 scores when the stool property was severely loose, 3 scores when the stool property was slight diarrhea, and 4 scores when the stool property was severe diarrhea; 0 score when the hemafecia degree was no hemafecia, 1 score when the hemafecia degree was slight occult blood, 2 scores when the hemafecia degree was severe occult blood, 3 scores when the hemafecia degree was slightly hematochezia visible to the naked eyes, and 4 scores when the hemafecia degree was severely hematochezia visible to the naked eye. The sum of the weight, the stool property and the hemafecia degree was the DAI score). PDE1i significantly reduced the severity of colitis. Two-tailed t-test was used to calculate the P-value, and * indicated that the P-value was less than 0.05. The above-mentioned experimental results show that the PDE1 inhibitor ITI214 could effectively alleviate colitis symptoms of mice, inhibits fibrosis and treats enteritis.

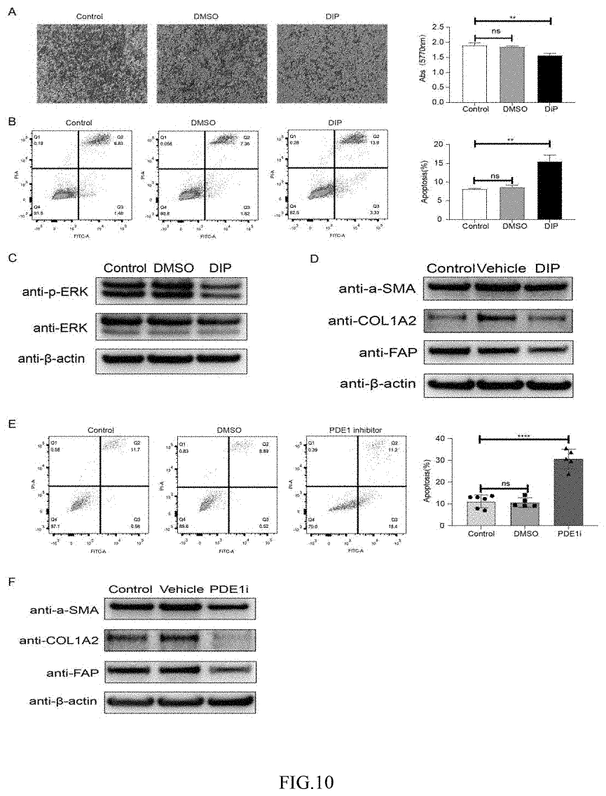

The effects of Dipyridamole and the PDE1 inhibitor ITI-214 on the proliferation and apoptosis of normal intestinal fibroblast CCD-18Co were detected. Cells were continuously stimulated with 20 μM pan PDE inhibitor DIP or 0.5 μM PDE1 inhibitor ITI-214, and the cells at 48 hours were collected respectively for flow analysis, crystal violet staining and protein extraction. Meanwhile, an intestinal interstitial fibrosis model was established in the CCD-18Co cell line, and 20 μM DIP or 0.5 μM PDE1 inhibitor ITI-214 was added one hour in advance. The CCD-18Co cells were continuously stimulated by TGF-β (10 ng/ml) and TNF-α (40 ng/ml), and the cells at 48 hours were collected to extract protein. The protein expression levels of α-SMA, COL1A2 and FAP were detected by Western blot. The proliferation and apoptosis of cells were detected by crystal violet staining and flow early/late apoptosis analysis. FIG. 10(A) and FIG. 10(C) showed that Dipyridamole inhibited the proliferation of fibroblasts; FIG. 10(B) showed that Dipyridamole promoted the apoptosis of fibroblasts; FIG. 10(D) showed that Dipyridamole inhibited the protein expression levels of α-SMA, COL1A2 and FAP in cells; FIG. 10(E) showed that ITI-214 promoted the apoptosis of fibroblasts; and FIG. 10(F) showed that ITI-214 inhibited the protein expression levels of α-SMA, COL1A2 and FAP in cells. The above experimental results showed that the pan PDE inhibitor Dipyridamole and the PDE1 inhibitor ITI214 could effectively inhibit the proliferation of fibroblasts, promote the apoptosis of fibroblasts, and inhibit the progress of fibrosis.

Molecular structure of the compound ITI214 was:

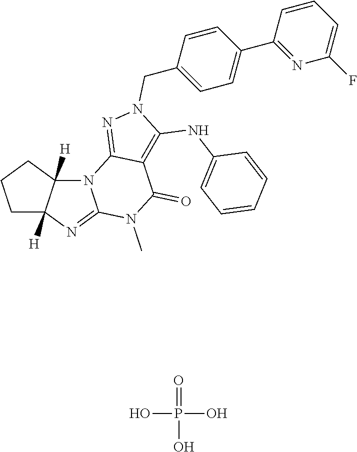 [SMILES: CN1C(=O)c2c(nn(Cc3ccc(-c4cccc(F)n4)cc3)c2Nc2ccccc2)N2C1=N[C@@H]1CCC[C@@H]12 ; O=P(O)(O)O]

### Example 8: Effect Experiment of PDE2 Inhibitor on Inhibiting Fibrosis and Treating Colitis

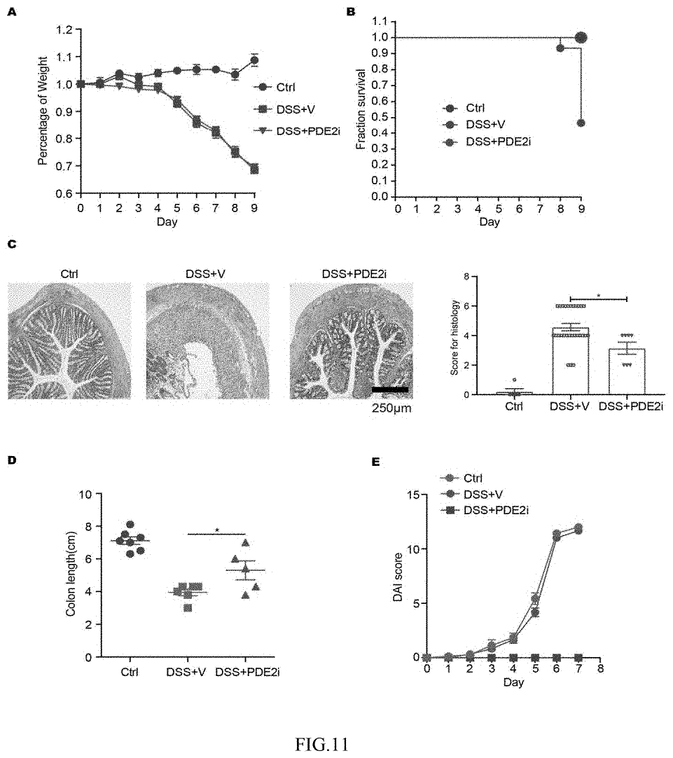

C57BL/6 mice were divided into three groups comprising normal control (Control, N=4), modeling+vehicle control (DSS+V, N=7, V represented vehicle, and DSS represented dextran sulfate sodium) and modeling+3 mg/kg PDE2 inhibitor PF-05085727 (DSS+PDE2i, N=7, PF-05085727, CAS No.: 1415637-72-7, purchased from Sigma, PZ0355) for experiment. The modeling method was as follows: on days 0-7, drinking water containing 2% DSS was fed every day, and changed into normal drinking from day 8. The drug and the vehicle were injected intraperitoneally on days 4-9, twice a day, and 100 μL each time. FIG. 11(A) showed the weight changes. PDE2i did not decrease significantly the weight. FIG. 11(B) showed the survival rate. PDE2i increased the survival rate of the disease from 40% to 100% (coincided with the control group). The left side of FIG. 11(C) was representative H&E staining pictures of colon tissue of each group, and the right side of FIG. 11(C) was pathological score of each group. It was found that, in comparison to the model group, the colonic tissue structure was significantly restored, the epithelial structure was more complete, the fibrosis was reduced, and inflammatory cells were reduced. FIG. 11(D) was the colon length. PDE2i significantly improved the colon length of mice with colitis. FIG. 11(E) showed the disease activity index score. PDE2i did not aggravate the activity degree of the disease. Two-tailed t-test was used to calculate the P-value, * indicated that the P-value was less than 0.05, and ** indicated that the P-value was less than 0.01. The above experimental results show that the PDE2 inhibitor PF-05085727 could alleviate DSS-induced acute mouse colitis symptoms to a certain degree.

Molecular structure of PDE2 inhibitor PF-05085727 (CAS No.: 1415637-72-7):

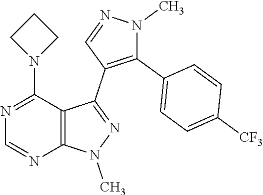 [SMILES: Cn1ncc(-c2nn(C)c3ncnc(N4CCC4)c23)c1-c1ccc(C(F)(F)F)cc1]

### Example 9: Effect Experiment of PDE9 Inhibitor on Inhibiting Fibrosis and Treating Colitis

C57BL/6 mice were divided into three groups comprising normal control (Control, N=4), modeling+vehicle control (DSS+V, N=7, DSS represented dextran sulfate sodium) and modeling+3 mg/kg PDE9 inhibitor PF-04447943 (DSS+PDE9i, N=7, PF-04447943, CAS No.: 1082744-20-4, purchased from MCE, HY-15441) for experiment. The modeling method was as follows: on days 0-10, drinking water containing 2% DSS was fed every day, and changed into normal drinking from day 11. The drug and the vehicle were injected intraperitoneally on days 4-12, twice a day, and 100 μL each time. FIG. 12(A) showed the weight changes. PDE9i significantly inhibited the weight loss from day 6. FIG. 12(B) showed the survival rate. PDE9i increased the survival rate of colitis from 40% to 100% (coincided with the control group). The left side of FIG. 12(C) was typical H&E staining pictures of colon, and the right side of FIG. 12(C) was pathological score. It was found that, in comparison to the model group, PDE9i could significantly restore the colonic tissue structure, the epithelial structure was more complete, the infiltration of inflammatory cells was decreased, and the fibrosis was reduced. FIG. 12(D) showed the colon length. PDE9i could significantly improve the colon length of mice with colitis. FIG. 12(E) showed the disease activity index score of mice. PDE9i significantly inhibited experimental colitis from day 6. Two-tailed t-test was used to calculate the P-value, * indicated that the P-value was less than 0.05, and ** indicated that the P-value was less than 0.01. The above experimental results showed that the PDE9 inhibitor PF-04447943 could effectively alleviate DSS-induced mouse colitis symptoms.

Molecular structure of PDE9 inhibitor PF-04447943 (CAS No.: 1082744-20-4):

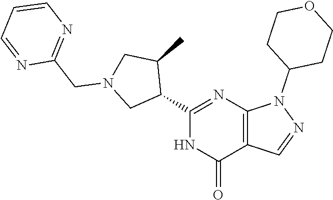 [SMILES: C[C@@H]1CN(Cc2ncccn2)C[C@H]1c1nc2c(cnn2C2CCOCC2)c(=O)[nH]1]

### Example 10: Effect Experiment of PDE3 Inhibitor on Inhibiting Fibrosis and Treating Colitis

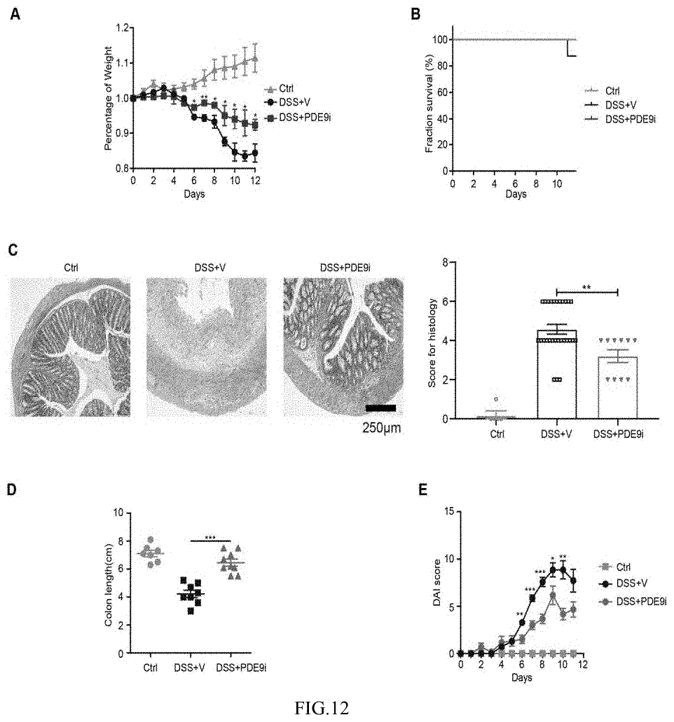

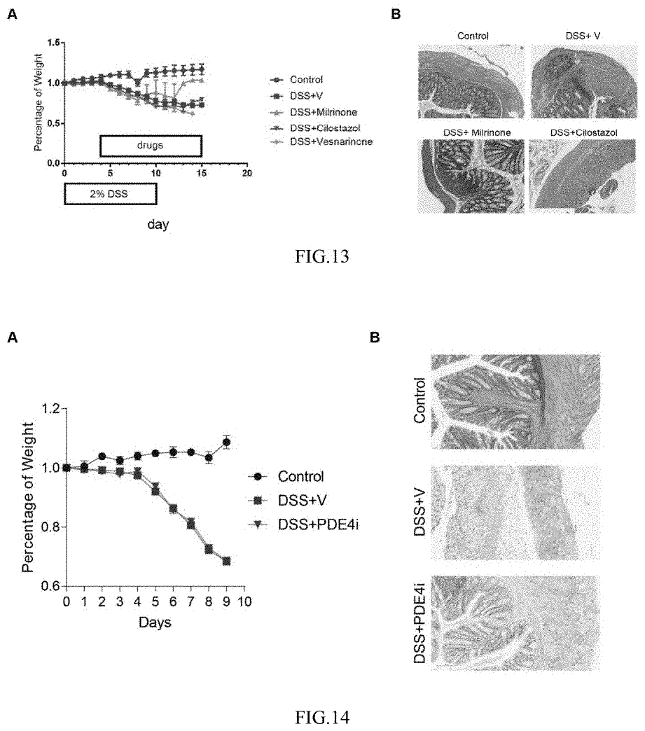

C57BL/6 mice were divided into five groups comprising normal control (Control, n=4), modeling+vehicle control (DSS+V, N=7, DSS represented dextran sulfate sodium), modeling+10 mg/kg Milirinone (CAS No.: 78415-72-2, purchased from MCE, HY-14252), modeling+10 mg/kg Cilostazol (CAS No.: 73963-72-1, purchased from MCE, HY-17464) and modeling+5 mg/kg Vesnarinone (CAS No.: 81840-15-5, purchased from MCE, HY-15297) for experiment. The modeling method was as follows: on days 0-10, drinking water containing 2% DSS was fed every day, and changed into normal drinking from day 11. The drug and the vehicle were injected intraperitoneally on days 4-15, twice a day, and 100 μL each time. FIG. 13(A) showed the weight changes. The weight did not decrease significantly. FIG. 13(B) was typical H&E staining pictures of colon, and it was found that, in comparison to the model group, the PDE3 inhibitor group could significantly restore the colonic tissue structure, the epithelial structure was more complete, the infiltration of inflammatory cells was decreased, and the fibrosis was reduced. The above experimental results showed that the PDE3 inhibitor could effectively improve DSS-induced mouse colitis symptoms. Milirinone, Cilostazol and Vesnarinone were all PDE3 inhibitors.

Molecular structure of Milirinone (CAS No.: 78415-72-2):

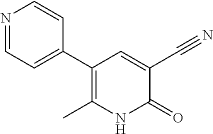 [SMILES: Cc1[nH]c(=O)c(C#N)cc1-c1ccncc1]

Molecular structure of Cilostazol (CAS No.: 73963-72-1):

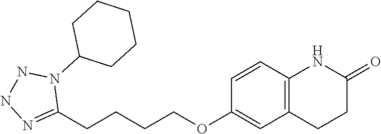 [SMILES: O=C1CCc2cc(OCCCCC3=NNNN3C3CCCCC3)ccc2N1]

Molecular structure of Vesnarinone (CAS No.: 81840-15-5):

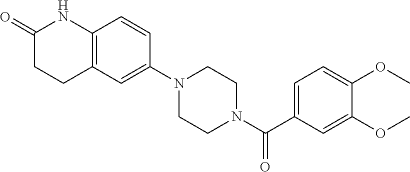 [SMILES: COc1ccc(C(=O)N2CCN(c3ccc4c(c3)CCC(=O)N4)CC2)cc1OC]

### Example 11: Experiment on Effect of PDE4 Inhibitor on Inhibiting Fibrosis and Treating Colitis

C57BL/6 mice were divided into three groups comprising normal control (Control, N=3), modeling+vehicle control (DSS+V, N=7, DSS represented dextran sulfate sodium) and modeling+3 mg/kg PDE4 inhibitor Roflumilast (DSS+PDE4i, N=7; Roflumilast, CAS No.: 162401-32-3, purchased from MCE, HY-15455) for experiment. The modeling method was as follows: on days 0-7, drinking water containing 2% DSS was fed every day, and changed into normal drinking from day 7. The drug and the vehicle were injected intraperitoneally on days 4-9, twice a day, and 100 μL each time. FIG. 14(A) showed the weight changes. PDE4i did not decrease significantly the weight. FIG. 14(B) was typical H&E staining pictures of colon, and it was found that, in comparison to the model group, the PDE4i inhibitor group could significantly restore the colonic tissue structure, the epithelial structure was more complete, the infiltration of inflammatory cells was decreased, and the fibrosis was reduced. The above experimental results showed that the PDE4 inhibitor Roflumilast could effectively alleviate DSS-induced mouse colitis symptoms.

Molecular structure of Roflumilast (CAS No.: 162401-32-3):

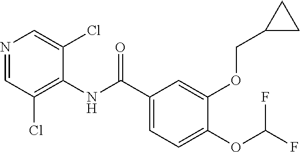 [SMILES: O=C(Nc1c(Cl)cncc1Cl)c1ccc(OC(F)F)c(OCC2CC2)c1]

### Example 12: Effect Experiment of PDE5 Inhibitor on Inhibiting Fibrosis and Treating Colitis

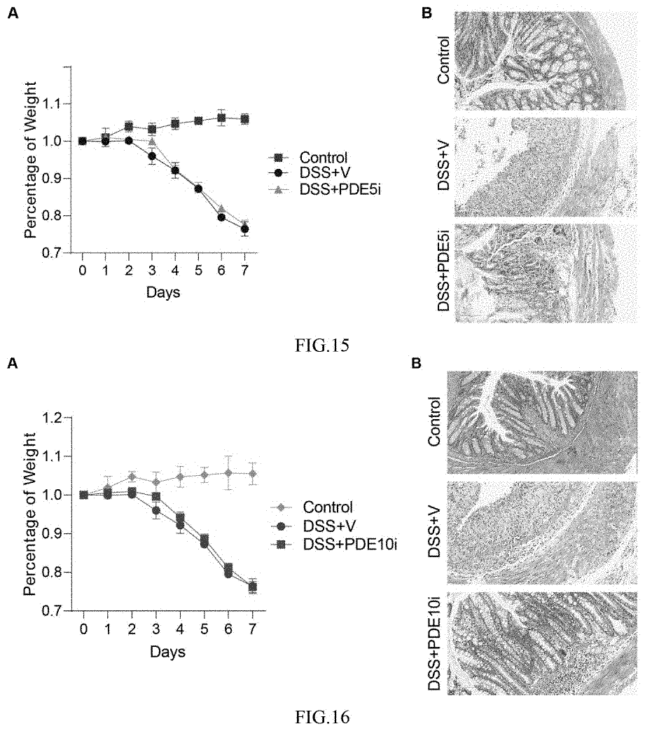

C57BL/6 mice were divided into three groups comprising normal control (Control, N=4), modeling+vehicle control (DSS+V, N=7, DSS represented dextran sulfate sodium) and modeling+50 mg/kg PDE5 inhibitor Icariin (DSS+PDE5i, N=7; Icariin, CAS No.: 489-32-7, purchased from MCE, HY-N0014) for experiment. The modeling method was as follows: on days 0-7, drinking water containing 2% DSS was fed every day, and changed into normal drinking from day 7. The drug and the vehicle were injected intraperitoneally on days 4-7, twice a day, and 100 μL each time. FIG. 15(A) showed the weight changes. PDE5i significantly inhibited the weight loss of colitis from day 6. FIG. 15(B) was a typical H&E staining picture of colon, and it was found that, in comparison to the model group, the PDE5 inhibitor Icariin group could significantly restore the colonic tissue structure, the epithelial structure was more complete, the infiltration of inflammatory cells was decreased, and the fibrosis was reduced.

Molecular structure of Icariin (CAS No.: 489-32-7):

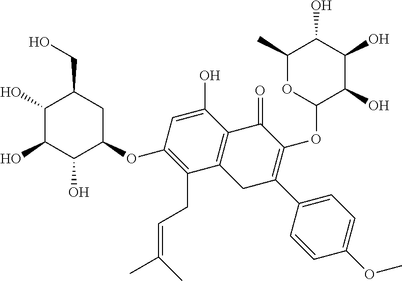 [SMILES: COc1ccc(C2=C(OC3O[C@@H](C)[C@H](O)[C@@H](O)[C@H]3O)C(=O)c3c(O)cc(O[C@@H]4C[C@H](CO)[C@@H](O)[C@H](O)[C@H]4O)c(CC=C(C)C)c3C2)cc1]

### Example 13: Effect Experiment of PDE10 Inhibitor on Inhibiting Fibrosis and Treating Colitis

C57BL/6 mice were divided into three groups comprising normal control (Control, N=3), modeling+vehicle control (DSS+V, N=7, DSS represented dextran sulfate sodium) and modeling+1 mg/kg PDE10 inhibitor Mardepodect hydrochloride (DSS+PDE10i, N=7; PF-04447943, CAS No.: 2070014-78-5, purchased from MCE, HY-50098A) for experiment. The modeling method was as follows: on days 0-7, drinking water containing 2% DSS was fed every day, and changed into normal drinking from day 7. The drug and the vehicle were injected intraperitoneally on days 4-7, twice a day, and 100 μL each time. FIG. 16(A) showed the weight changes. PDE10i did not decrease the weight significantly. FIG. 16(B) was typical H&E staining pictures of colon, and it was found that, in comparison to the model group, the PDE10i group could significantly restore the colonic tissue structure, the epithelial structure was more complete, the infiltration of inflammatory cells was decreased, and the fibrosis was reduced. The above experimental results showed that the PDE10 inhibitor Mardepodect hydrochloride could effectively alleviate DSS-induced mouse colitis symptoms.

Molecular structure of Mardepodect hydrochloride (CAS No.: 2070014-78-5):

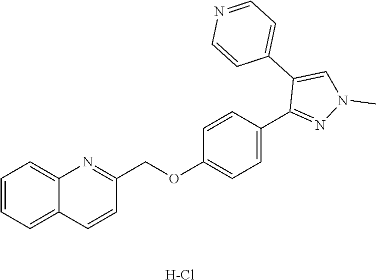 [SMILES: Cn1cc(-c2ccncc2)c(-c2ccc(OCc3ccc4ccccc4n3)cc2)n1 ; "H-Cl"]

The present disclosure includes various changes in the scope thereof, and these changes do not depart from the scope of the present disclosure. In addition, all the modifications that are obvious to those skilled in the art are included in the scope of the claims of the present disclosure.

1. A method of preventing and/or treating a fibrotic disease, comprising administering a therapeutically effective amount of a PDE inhibitor or a pharmaceutically acceptable salt thereof to a subject in need thereof.

2. The method according to claim 1, wherein the PDE inhibitor is at least one selected from the group consisting of pan PDE inhibitors, PDE1 inhibitors, PDE2 inhibitors, PDE3 inhibitors, PDE4 inhibitors, PDE5 inhibitors, PDE6 inhibitors, PDE7 inhibitors, PDE8 inhibitors, PDE9 inhibitors, PDE10 inhibitors, and PDE11 inhibitors.

3. (canceled)

4. The method according to claim 1, wherein the PDE inhibitor is at least one selected from the group consisting of Dipyridamole, ITI214, PF-05085727, PF-04447943, Milirinone, Cilostazol, Vesnarinone, Roflumilast, Icariin, and Mardepodect hydrochloride.

5. The method according to claim 1, wherein the fibrotic disease is selected from fibrotic diseases of liver, gallbladder, lung, kidney, bladder, heart, blood vessel, eye, skin, pancreas, gastrointestinal, bone marrow, penis, breast, and muscle; preferably, the fibrotic disease is selected from fibrotic diseases of liver, gallbladder, lung and gastrointestinal.

6. (canceled)

7. The method according to claim 1, wherein the fibrotic disease is selected from cirrhosis, hepatic fibrosis, liver injury, hepatic failure and biliary atresia; or the fibrotic disease is selected from idiopathic pulmonary fibrosis, silicosis, cystic fibrosis and pulmonary hypertension; the fibrotic disease is selected from fibrosis of stomach, duodenum, small intestine and colon.

8.-9. (canceled)

10. A combination drug for a fibrotic disease, comprising the PDE inhibitor or the pharmaceutically acceptable salt thereof according to claim 1, other active ingredients, and a pharmaceutically acceptable carrier.

11. The method according to claim 1, wherein the subject is selected from children, adults or the elderly, preferably selected from children; preferably, the children are administered once a day or multiple times a day, with a dosage of 0.1-100 mg/kg/day, more preferably 3-20 mg/kg/day; preferably, the PDE inhibitor is administered in the form of a pharmaceutical composition, and the content of the PDE inhibitor in the pharmaceutical composition is 0.1 wt % to 100 wt %.

12. A method of preventing and/or treating a fibrotic disease, comprising administering a therapeutically effective amount of a PDE inhibitor or a pharmaceutically acceptable salt thereof to a subject in need thereof, wherein the fibrotic disease is gastrointestinal fibrosis, and the PDE inhibitor is at least one selected from the group consisting of pan PDE inhibitors, PDE1 inhibitors, PDE2 inhibitors, PDE3 inhibitors, PDE4 inhibitors, PDE5 inhibitors, PDE6 inhibitors, PDE7 inhibitors, PDE8 inhibitors, PDE9 inhibitors, PDE10 inhibitors, and PDE11 inhibitors.

13. The method according to claim 12, wherein the fibrotic disease is selected from fibrosis of stomach, duodenum, small intestine and colon; preferably, the fibrotic disease is colitis or fibrosis of colitis.

14. The method according to claim 12, wherein the PDE inhibitor is at least one selected from the group consisting of Dipyridamole, ITI214, PF-05085727, PF-04447943, Milirinone, Cilostazol, Vesnarinone, Roflumilast, Icariin, and Mardepodect hydrochloride.

15. The method according to claim 12, wherein the subject is selected from children, adults or the elderly, preferably selected from children; preferably, the children are administered once a day or multiple times a day, with a dosage of 0.1-100 mg/kg/day, more preferably 3-20 mg/kg/day; preferably, the PDE inhibitor is administered in the form of a pharmaceutical composition, and the content of the PDE inhibitor in the pharmaceutical composition is 0.1 wt % to 100 wt %.

16. The method according to claim 12, wherein the pharmaceutically acceptable salt of the PDE inhibitor is sodium salt or inorganic acid salt of the PDE inhibitor; preferably, the pharmaceutically acceptable salt of the PDE inhibitor is Dipyridamole Sodium Chloride or Dipyridamole hydrochloride.

17. The method according to claim 12, wherein the PDE inhibitor or the pharmaceutically acceptable salt thereof is administered by intravenous administration, intramuscular administration, subcutaneous administration, intraorgan administration, nasal administration, intradermal administration, drop, intracerebral administration, intrarectal administration, vagina administration, intraperitoneal administration, intratumor administration, tumor proximal end administration, or lesion administration; preferably, the PDE inhibitor or the pharmaceutically acceptable salt thereof is prepared into oral administrations or non-oral administrations, comprising tablets, pills, powders, granules, soft capsules, hard capsules, microcapsules, lozenges, syrups, liquids, emulsions, suspensions, controlled-release preparations, sustained-release preparations, aerosols, films, injections, transdermal absorption preparations, creams, ointments, lotions, adhesive preparations, suppositories, medicinal granules.

18. A combination drug for a fibrotic disease, comprising the PDE inhibitor or the pharmaceutically acceptable salt thereof according to claim 12, other active ingredients, and a pharmaceutically acceptable carrier, wherein the fibrotic disease is gastrointestinal fibrosis.

19. A method of preventing and/or treating a fibrotic disease, comprising administering a therapeutically effective amount of a PDE inhibitor or a pharmaceutically acceptable salt thereof to a subject in need thereof, wherein the fibrotic disease is fibrosis of liver and/or gallbladder, and the PDE inhibitor is at least one selected from the group consisting of pan PDE inhibitors, PDE1 inhibitors, PDE2 inhibitors, PDE3 inhibitors, PDE4 inhibitors, PDE5 inhibitors, PDE6 inhibitors, PDE7 inhibitors, PDE8 inhibitors, PDE9 inhibitors, PDE10 inhibitors, and PDE11 inhibitors.

20. The method according to claim 19, wherein the fibrotic disease is selected from cirrhosis, hepatic fibrosis, liver injury, hypohepatia and biliary atresia.

21. The method according to claim 19, wherein the PDE inhibitor is at least one selected from the group consisting of Dipyridamole, ITI214, PF-05085727, PF-04447943, Milirinone, Cilostazol, Vesnarinone, Roflumilast, Icariin, and Mardepodect hydrochloride.

22. The method according to claim 19, wherein the subject is selected from children, adults or the elderly, preferably selected from children; preferably, the children are administered once a day or multiple times a day, with a dosage of 0.1-100 mg/kg/day, more preferably 3-20 mg/kg/day; preferably, the PDE inhibitor is administered in the form of a pharmaceutical composition, and the content of the PDE inhibitor in the pharmaceutical composition is 0.1 wt % to 100 wt %.

23. The method according to claim 19, wherein the PDE inhibitor or the pharmaceutically acceptable salt thereof is administered by intravenous administration, intramuscular administration, subcutaneous administration, intraorgan administration, nasal administration, intradermal administration, drop, intracerebral administration, intrarectal administration, vagina administration, intraperitoneal administration, intratumor administration, tumor proximal end administration, or lesion administration; preferably, the PDE inhibitor or the pharmaceutically acceptable salt thereof is prepared into oral administrations or non-oral administrations, comprising tablets, pills, powders, granules, soft capsules, hard capsules, microcapsules, lozenges, syrups, liquids, emulsions, suspensions, controlled-release preparations, sustained-release preparations, aerosols, films, injections, transdermal absorption preparations, creams, ointments, lotions, adhesive preparations, suppositories, medicinal granules.

24. A combination drug for a fibrotic disease, comprising the PDE inhibitor or the pharmaceutically acceptable salt thereof according to claim 19, other active ingredients, and a pharmaceutically acceptable carrier, wherein the fibrosis disease is fibrosis of liver and/or gallbladder.
